(19) United States
(12) Patent Application Publication
(10) Pub. No.: US 2021/0393807 A1
(43) Pub. Date: Dec. 23, 2021

(54) PHOTOACOUSTIC VOLTAGE-SENSITIVE DYES FOR IN VIVO IMAGING

(71) Applicants: The Johns Hopkins University, Baltimore, MD (US); University of Connecticut, Farmington, CT (US)

(72) Inventors: Dean F. Wong, Lutherville, MD (US); Emad Boctor, Baltimore, MD (US); Haichong Zhang, Baltimore, MD (US); Jeeun Kang, Baltimore, MD (US); Jin Kang, Glenelg, MD (US); Arman Rahmim, Baltimore, MD (US); Dan Thorek, Baltimore, MD (US); Leslie Loew, West Hartford, CT (US); Ping Yan, Avon, CT (US)

(21) Appl. No.: 17/194,649

(22) Filed: Mar. 8, 2021

Related U.S. Application Data
(63) Continuation of application No. 15/837,891, filed on Dec. 11, 2017, now abandoned.
(60) Provisional application No. 62/432,109, filed on Dec. 9, 2016.

(51) Int. Cl. A61K 49/22; C09B 23/08; A61B 5/00; A61K 49/00; C09B 23/01

(52) U.S. Cl. CPC A61K 49/22; C09B 23/086; C09B 23/0066; A61K 49/0032; A61B 5/0095

(57) ABSTRACT

Photoacoustic voltage dependent dyes and their use in measuring an electrophysiological activity in a subject in vivo are disclosed.

### CROSS-REFERENCE TO RELATED APPLICATIONS

This application is a continuation of U.S. patent application Ser. No. 15/837,891, filed Dec. 11, 2017, which claims the benefit of U.S. Provisional Application No. 62/432,109, filed Dec. 9, 2016, each of which is incorporated herein by reference in its entirety.

### FEDERALLY SPONSORED RESEARCH OR DEVELOPMENT

This invention was made with government support under EB001963 and MH106083 awarded by the National Institutes of Health. The government has certain rights in the invention.

### BACKGROUND

The quantification of neurotransmitter (NT) activity with high temporal resolution is essential to build a comprehensive map of brain function. To achieve this goal, the low temporal resolution, but high pharmacological specificity, of PET and higher spatial and temporal resolution, but less specific, MRI must be improved. Membrane potential measurements, utilizing direct electrical recording or by imaging using voltage-sensitive dyes (VSDs), have been used to observe spontaneous NT events by means of voltage fluctuations caused by ionic currents. Imaging approaches have the advantage, in general, that patterns of activity can be studied with high resolution over large areas of brain. (Canepari, et al., 2015) Purely optical based detection approaches, however, have several limitations, which include a small dynamic range and only shallow penetration depth due to light scattering and absorbance of overlying tissue in vivo. Photoacoustic (PA) imaging is an emerging hybrid imaging modality. In PA imaging, a noninvasive molecular light absorbance dependent acoustic signal occurs at depths of up to several centimeters in biological tissue, (Wang, et al., 2012; Wang, 2009) with a micro to millimeter spatial resolution that is limited by the acoustic bandwidth and focusing. The mechanism behind PA imaging is that upon excitation by a short-pulsed laser, thermal relaxation of the chromophore excited state induces local thermal elastic expansion. Using near-infrared laser excitation and ultrasound detection enhances the light-penetration and resulting imaging depth by minimizing the absorptive and scattering attenuation during the light propagation through the biological tissue. Toward use of this technique for functional brain imaging, previous research has demonstrated PA imaging to be capable of monitoring brain activity based on the blood-oxygen-level dependent signal change. This imaging procedure, which does not rely on the administration of an exogenous contrast agent, (Hu, 2016; Nasiriavanaki, et al., 2014; Yao, et al., 2014) is susceptible to several nonlinear physiological and biophysical parameters in addition to the NT activity or electrical signaling. As a result, it is regarded as an indirect, semiquantitative reflection of the membrane potential change in neurons. (Arthurs, et al., 2007; Arthurs, et al., 2002) An alternative approach is needed to provide direct readout of membrane potential events in cerebral tissues. A number of contrast agents have been previously evaluated for use with PA imaging to selectively visualize tumor tissue or metabolic properties. (Weber, et al., 2016; Wu, et al., 2014; Luke, et al., 2012) Most of the proposed PA contrast agents have been based on the extinction coefficient of the compound used, as the materials with a stronger absorbance should provide strong PA intensity. The presently disclosed subject matter demonstrates how voltage-dependent PA signals may be produced by VSDs. The theoretical concept is first developed whereby the fluorescence quenching of the voltage-dependent dye leads to a reciprocal enhancement of PA intensity. Based on this concept, a near-infrared PA-VSD (PAVSD800-2), whose PA intensity change is sensitive to membrane potential, was synthesized. The performance of the PA-VSD developed was tested with a lipid vesicle test system that allowed the membrane potential to be readily manipulated and both the PA and spectrophoto/fluorometric response to be measured. Importantly, it has near-infrared excitation and emission bands, which would make it appropriate for deep NT activity imaging applications. Furthermore, the theoretical model based on the photophysical properties of the VSD enables the PA voltage sensitivity to be quantitatively estimated. Further development of the ideas described herein promises exogenous contrast agents with high temporal and spatial resolution for deep brain NT activity measurements.

### SUMMARY

In some aspects, the presently disclosed subject matter provides a photoacoustic voltage dependent dye of formula (I):

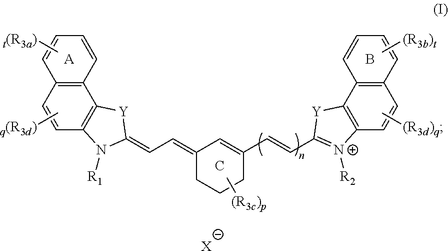

wherein: n is an integer selected from 0, 1, and 2; p and t are each independently integers selected from 1, 2, 3, and 4; q is an integer selected from 1 or 2; A and B can be present or absent; C is a ring that can be present or absent and when present forms part of the polymethine chain, wherein ring C or the polymethine chain of which it forms a part of can be substituted with R_{3c}; R₁ and R₂ can be the same or different and are each independently selected from alkyl, substituted alkyl, and —(CH₂CH₂O)ₘ—R₄, wherein m is an integer from 1 to 20 and R₄ is selected from the group consisting of H, alkyl, and —OR₅, wherein R₅ is alkyl; each R₃ₐ, R_{3b}, R_{3c}, and R_{3d} is selected from the group consisting of alkyl, halogen, hydroxyl, cyano, and alkoxyl; Y is selected from O, S, and C(R₆)₂, wherein each R₆ is independently H or alkyl; X is a counterion; and pharmaceutically acceptable salts thereof.

In other aspects the presently disclosed subject matter provides a method for measuring an electrophysiological activity in a subject in vivo, the method comprising administering one or more photoacoustic voltage dependent dyes of formula (I) to a target area of the subject; irradiating the target area of the subject with near-infrared radiation; and measuring a photoacoustic signal from the target area of the subject, wherein the photoacoustic signal is indicative of the electrophysiological activity in the subject.

In particular aspects, the presently disclosed method further comprises co-administering the dye with an agent capable of pharmacological modulation of adenosine receptor signaling, incorporating the dye in a brain-penetrating nanoparticle, incorporating the dye in a microbubble, focused ultrasound, and combinations thereof.

In yet other aspects, the presently disclosed subject matter provides an integrated photoacoustic imaging system for measuring an electrophysiological activity in a subject in vivo, the system comprising: (a) a near-infrared light source; (b) an ultrasound probe; and (c) a data acquisition system.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 demonstrates the principle of the fluorescence quenching effect on a representative VSD, e.g., (PAVSD800-2) according to a varying membrane potential: typically −70 and 0 mV in the polarized/depolarized states of neurons, respectively;

FIG. 2A, FIG. 2B, and FIG. 2C show the PAVSD800-2 synthetic scheme. Conditions: (FIG. 2A) CH₃CN, 130° C., 14 h, 64%; (FIG. 2B) Ac₂O, 130° C., 1 h; and (FIG. 2C) 1:1 Ac₂O/pyridine, 100° C., 40%;

FIG. 3 shows a phantom experiment setup for PA characterization of PAVSD800-2. Near-infrared light excited the sample in the tubing (green) through the optical fiber bundle, and the generated PA signals were captured by a clinical linear array transducer (10-MHz center frequency);

FIG. 4A, FIG. 4B, and FIG. 4C show the spectrophotometric characteristics of the (FIG. 4A and FIG. 4B) PAVSD800-2 and (FIG. 4C) the di-SC2(5) at the simulated resting/action states using valinomycin (Val) and gramicidin (Gra). (FIG. 4A) Absorbance spectrum of the dye PAVSD800-2 for the concentration of 6 μM. (FIG. 4B) Absorbance spectra at the wavelength of 800 nm for different dye concentrations. (FIG. 4C) Absorbance spectrum of the dye di-SC2(5) for the concentration of 6 μM;

FIG. 5A, FIG. 5B, FIG. 5C, and FIG. 5D show the spectrofluorometric characteristics of the (FIG. 5A, FIG. 5B, and FIG. 5C) PAVSD800-2 and (FIG. 5D) the di-SC2(5). (FIG. 5A) The emission spectrum of the dye PAVSD800-2 at 6 μM concentration, where vesicles prepared in K⁺ buffer are diluted into isotonic Na⁺ buffer to generate a membrane potential upon addition of valinomycin. (FIG. 5B) Negative control, where K⁺ buffer is used for both the inside and outside. (FIG. 5C) The fluorescence emission at 820 nm for different concentrations. (FIG. 5D) Fluorescence emission spectrum of the dye, di-SC2(5) at 6 μM concentration;

FIG. 6 shows the fractional change of absorbance at 800 nm and fluorescence signal at 825 nm between the resting and action states by adding valinomycin. The fluorescence quenching effect was significantly larger at all concentrations than the absorption change (eight times stronger at 9 μM concentration);

FIG. 7A, FIG. 7B, and FIG. 7C show (FIG. 7A) PA spectra of the PA-VSD for different concentrations at the prestimulus (black) and stimulated polarized (red) and depolarized (green) states using valinomycin (Val) and gramicidin (Gra). (FIG. 7B) Voltage dependent PA intensity at 800 nm for different concentrations. (FIG. 7C) PA intensity changes at varying PA-VSD concentrations for polarized (Val, red) and depolarized (Gra, green) states relative to prestimulus (VSD, black). Error bars are standard deviation;

FIG. 8A, FIG. 8B, and FIG. 8C show the PA images for three conditions: the depolarized state from the (FIG. 8A) PAVSD800-2 only, (FIG. 8B) the valinomycin-induced polarized state, and (FIG. 8C) the gramicidin-reinduced depolarized state;

FIG. 9A and FIG. 9B show the stimulated resting and action state contrast change relative to the prestimulus state intensity. (FIG. 9A) Image produced by subtraction of the gramicidin data from the initial PA dye image is considered a negative control and (FIG. 9B) the difference between the initial PA dye image and the valinomycin-induced polarized state. Magnitude represents the fractional change of the intensity relative to the pre-stimulus state intensity;

FIG. 10 shows the theoretical PA signal change based on the absorbance change and fluorescence change from the experimental data;

FIG. 11A, FIG. 11B, FIG. 11C, and FIG. 11D show the transcranial VSD sensing setup using PA imaging system: (FIG. 11A) schematic diagram of experimental setup; (FIG. 11B) absorbance spectra of VSD, deoxy- and oxy-hemoglobin. Dotted line indicates the wavelength used in in vivo experiment, i.e., 790 nm; (FIG. 11C) cross-sectional PA image of cerebral cortex; (FIG. 11D) in vivo experimental protocol. SSS: Superior sagittal sinus; SCV: Superior cortical veins. SSS: superior sagittal sinus; SCV: superior cortical veins; L-MC/R-MC: left/right motor cortex. Note that the outlines for brain and motor cortex in FIG. 11C was drawn based on the rat brain atlas (Interaural 11.2 mm, Bregma 2.2 mm, FIG. 15) (Paxinos, et al., 2014);

FIG. 12A and FIG. 12B show phantom experimental results using lipid vesicle membrane model with VSD. (FIG. 12A) Absorbance and fluorescence emission spectrum of near-infrared VSD. (FIG. 12B) Near-infrared photoacoustic spectrum and intensity change at the peak absorbance, i.e., 790 nm (P<0.0465 for VSD vs. VSD+Val; P<0.0444 for VSD+Val vs. VSD+Val+Gra). Val: valinomycin; Gra:gramicidin;

FIG. 13A, FIG. 13B, and FIG. 13C show the representative PA sensing of electrophysiological neural activity: (FIG. 13A) time-averaged neural activity maps and STFT spectrograms of VSD response of each protocol; and (FIG. 13B) neural activity index over 10 min in seizure, control, and negative control groups. Note that the regions-of-interest in the STFT spectrograms are indicated with asterisk marks in the respective neural activity map. The representative temporal evolution of neural activity map over time for seizure, control, and negative control groups also was measured (data not shown). Also, the neural activity index for each rat (i.e., rat 1, 4, and 5) is presented in FIG. 18. The neural activity index during seizure has been significantly differentiated from that in baseline phase (P<0.0001). White bar in FIG. 13A indicates 1 mm;

FIG. 14A and FIG. 14B shows the evolution of EEG signal in the in vivo protocol identical to transcranial PA imaging: (FIG. 14A) Representative EEG traces recorded from rat motor cortex before and during induction of status epilepticus using chemoconvulsant PTZ. The baseline and control EEG traces represent EEG activity in an anesthetized rat (see methods) with and without IR780+lexiscan given at the dosage found to not alter baseline EEG activity in the pilot study. PTS seizure induction proceeded in classical style described previously wherein episodic epileptiform burst activity evolved into status epilepticus with intermittent occurrence of seizures and stable interictal activity. (FIG. 14B) EEG spectral quantitation of the EEG recording done every 10 sec epoch during the EEG showed the expected progression in EEG power associated with evolution of the PTZ induced status epilepticus. Time line of PTZ injections indicated with arrows. Expanded EEG traces on top show the uniform epileptiform discharges after following second PTZ injection and below a seizure event followed of post-ictal suppression indicating the termination of that event;

FIG. 15A and FIG. 15B are diagrams of a rat brain atlas for representative regions-of-interest: (FIG. 15A) coronal plane at interaural 11.2 mm and bregma 2.2 mm, (FIG. 15B) horizontal plane at lateral 1.9 mm. Note that the motor cortex, the target cortex-of-interest, are highlighted by red color (Prior Art, Paxinos (2014));

FIG. 16 shows the estimated quantum yield of near-infrared VSD: The equation shown previously in Zhang, et al. (2017), was employed for the estimation of quantum yield using the fractional change of PA intensity and spectrophotometric measurements obtained by lipid vesicle experiment mimicking polarized/depolarized cell state (FIG. 12);

FIG. 17A, FIG. 17B, FIG. 17C, and FIG. 17D show normalized time-frequency analysis method: (FIG. 17A) Flow chart of the proposed signal analysis; (FIG. 17B) Example of STFT segments in frequency and time domain; (FIG. 17C) Spatial-projected spectrogram for specific motor cortex region of interest; (FIG. 17D) Frequency-projected neural activity map for entire field-of-view. The white bar in FIG. 17C indicates 1 mm. (X, Y): lateral and axial indexes of a segment in PA image; (x, y): lateral and axial pixel indexes in each segment;

FIG. 18 shows individual neural activity index for each rat in seizure, control, and negative control;

FIG. 19 shows fractional changes of PA intensity depending on Adenosine receptor signaling modulation using intravenous regadenoson administration. Each PA sequences, i.e., VSD only and VSD+regadenoson, was measured from the brain tissue region (3 mm below the skin surface), and projected during last 2 min (8-10 min, 480 times points);

FIG. 20A, FIG. 20B, and FIG. 20C show minimal correlation projection (MCP) image using cross-correlation coefficients with varying time interval, i.e., 0.25 sec, 0.5 sec, and 1 sec, which respectively corresponds to 1, 2, 4 frame intervals with the imaging rate at 4 frames per second. (FIG. 20A) region of interest for the inter-frame cross-correlations, (FIG. 20B) MAP images of baseline (PTZ−, VSD−) and seizure groups (PTZ+, VSD−) for brain tissue region. (FIG. 20C) Cross-correlation coefficient for varying time intervals;

FIG. 21A, FIG. 21B, and FIG. 21C show VSD toxicity study using EEG recordings during direct cortical applications using a cranial window in rats. (FIG. 21A) Schematic of experimental protocol. A rectangular cranial window drilled under anesthesia overlying unilateral motor cortex. Duramater was kept intact. Following craniotomy, a small window was made in duramater without traversing blood vessels. (FIG. 21B) EEG recording of baseline brain activity under anesthesia was followed by using a Hamilton micro syringe to apply increasing concentrations of IR780 directly to the cortical surface via window made in duramater. Base EEG remained unaltered at lower concentrations but showed significant background suppression after applying a 100× solution. This study allowed the concentration of IR780 10× for all PA experiments to be determined. (FIG. 21C) EEG power spectral quantification for every 10-sec epoch of EEG over the duration of the recording confirmed EEG suppression with the 100× dose;

FIG. 22A and FIG. 22B show a representative in vivo experimental protocol for the correlation between photoacoustic recording and microdialysis;

FIG. 23 shows the photoacoustic time-averaged neural activity map for the in vivo experimental protocol described in Example 3;

FIG. 24 shows the correlation between photoacoustic recording and glutamate concentration of extracellular fluid samples during different time points of the experiment;

FIG. 25 shows experimental results from Example 3;

FIG. 26 shows NMDA agonist-induced simultaneous glutamate release by microdialysis;

FIG. 27 shows EEG power at baseline and during NMDA infusion;

FIG. 28 shows a representative in vivo experimental protocol for visual stimulation and photoacoustic neural activing recording;

FIG. 29 shows representative in vivo experimental results of the correlation between photoacoustic and microdialysis;

FIG. 30A, FIG. 30B, and FIG. 30C show preliminary in vivo experimental results of transcranial photoacoustic sensing using a LED light source: (FIG. 30A) is a representative experimental configuration for a neonatal piglet model; (FIG. 30B) is a photoacoustic image and its superimposition on corresponding ultrasound image; and FIG. 30C is an estimation of oxygen saturation;

FIG. 31A is a simplified schematic of a representative photoacoustic/fluorescence imaging system for imaging through intact skull;

FIG. 31B is a CAD design of a representative probe for both laser excitation and imaging; and

FIG. 32 depicts the integration of a focused ultrasound ring element, with a 2D ultrasound array with 2-3 mm hole, e.g., 2-3 mm hole, in the middle carrying, for example, 100K multi-modal fibers.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Photoacoustic Voltage-Sensitive Dyes for In Vivo Imaging

Voltage-sensitive dyes (VSDs) are generally designed to monitor membrane potential by detecting fluorescence changes in response to neuronal or muscle electrical activity. Fluorescence imaging, however, is limited by depth of penetration and high scattering losses, which leads to low sensitivity in in vivo systems for external detection. By contrast, photoacoustic (PA) imaging, an emerging modality, is capable of deep tissue, noninvasive imaging by combining near-infrared light excitation and ultrasound detection. The presently disclosed subject matter demonstrates that voltage dependent quenching of dye fluorescence leads to a reciprocal enhancement of PA intensity. A near-infrared photoacoustic VSD (PA-VSD), whose PA intensity change is sensitive to membrane potential, was synthesized. In the polarized state, this cyanine-based probe enhances PA intensity while decreasing fluorescence output in a lipid vesicle membrane model. A theoretical model accounts for how the experimental PA intensity change depends on fluorescence and absorbance properties of the dye. These results not only demonstrate PA voltage sensing, but also emphasize the interplay of both fluorescence and absorbance properties in the design of optimized PA probes. Together, these results demonstrate PA sensing as a potential new modality for recording and external imaging of electrophysiological and neurochemical events in the brain.

### A. Theory and Principle

Design of a Photoacoustic Voltage-Sensitive Dye Based on Photophysics and Photochemistry

When a chromophore absorbs a photon to occupy an excited state, it can relax back to the ground state either by emitting a photon or by shedding its energy as heat. The former is termed “radiative decay” and has a rate, kᵣ, that depends on the chromophore and its molecular environment; the radiative decay is measured as fluorescence. The thermal decay route, with a rate kₜ, also depends on both the structure of the dye molecule and its environment. Specifically, low energy internal vibrational modes can facilitate thermal relaxation; and the environment, including interactions with solvent or with specific interacting partners—quenchers—also offer nonradiative decay pathways. The PA intensity depends on the thermal decay of chromophores after they are excited by a short intense laser pulse; the resultant rapid and large thermal decay produces a burst of heat that locally increases the kinetic energy of neighboring molecules and can be detected with an ultrasound detector. The key point is that for a given chromophore, the efficiency of thermal (th) (acoustic) and radiative [fluorescence (F) and phosphorescence] decay processes are in competition; for most organic chromophores, phosphorescence can be neglected, leading to simple relationships:

$${Φ}_{p} = \frac{{k}_{r}}{{k}_{r} + {k}_{1}} . {Φ}_{ph} = \frac{\text{?}}{\text{?} + \text{?}} . \\ \text{?} \text{indicates text missing or illegible when filed} \qquad ( 1 )$$

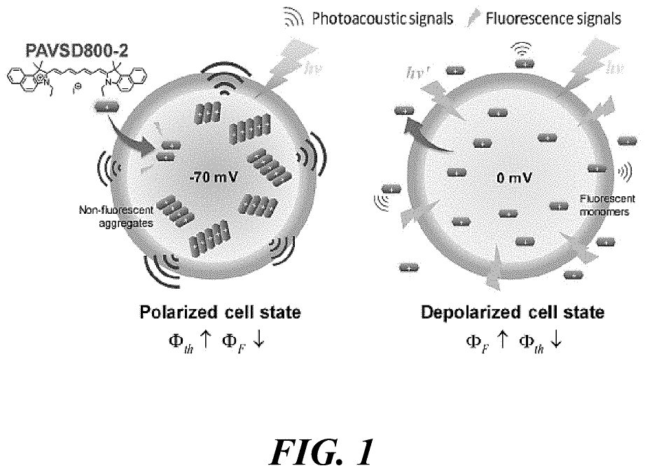

These equations give the theoretical quantum efficiency, Φ, for fluorescence and PAs, corresponding, respectively, to the probability that an absorbed photon will be transformed into an emitted photon, detectable as fluorescence, or into a thermoelastic expansion of an absorber, detectable as PA. Both arithmetically and by the principle of conservation of energy, the sum of Φ_{F} and Φₜₕ must be unity. Indeed, there is experimental evidence of the reciprocal relationship between fluorescence efficiency and PA efficiency. For example, Qin et al. (Qin, et al., 2015) designed a PA contrast agent, in which PA intensity was enhanced by suppressing the fluorescence emission. It has long been known that cyanine dyes have a tendency to form aggregates at high concentrations. The aggregates are nonfluorescent. Waggoner et al. developed a series of highly sensitive VSDs based on cyanine dyes that, because they have delocalized positive charge, redistributed across cell membranes as a function of the membrane potential. (Sims, et al., 1974) Because the charge is delocalized, they are able to permeate through the hydrophobic cell membrane and redistribute according to the Nernst equation. The idea is that the dye molecules will accumulate inside polarized cells at sufficiently high local concentration to produce nonfluorescent aggregates; upon depolarization, the dye molecules will be released and diluted into the larger external volume, favoring fluorescent monomers (FIG. 1). Indeed, under the right circumstances, depolarization can produce >100 fold increases in cyanine dye fluorescence, (Loew, et al., 1985) because the tendency for the dyes to aggregate shifts the equilibrium for more monomeric dye to be driven in by the membrane voltage. Without wishing to be bound to any one particular theory, it was thought that there should be a reciprocal relationship between the fluorescence and the PA sensitivity to membrane potential (FIG. 1) because the aggregated state of the cyanine dyes should favor thermal decay of the excited state. However, previously reported cyanine dyes with superior voltage sensitivity, such as di-SC2, (Loew, et al., 1985) absorb around 650 nm or lower. Because the best available laser systems for PA detection are in the near-infrared spectral range and because longer wavelengths allow deeper penetration into tissue, a cyanine dye was designed and synthesized specifically for application as PA-VSD. One particular candidate, PAVSD800-2, shows a fluorescence emission in the near-infrared region with the absorbance peak around 800 nm and fluorescence emission at 828 nm in the presence of lipid membranes.

Thermal Confinement and Fluorescence Emission

To be able to quantitatively predict the PA response to a change in membrane potential, a more detailed mathematical model was derived to incorporate the PA signal enhancement with the fluorescence quenching effect. This model also accounts for possible changes in dye absorbance. In the classic formulation, the initial PA pressure, p₀, has been modeled based on absorbance

$${p}_{0} = \frac{β}{ρ \text{?} x} {Φ}_{th} \text{?} Υ = {ΓΦ}_{th} \text{?} Υ , \\ \text{?} \text{indicates text missing or illegible when filed} \qquad ( 2 )$$

where β is the thermal compressibility; ρ is the mass density; Cᵥ is the heat capacity; κ is the isothermal compressibility; Φₜₕ is the thermomechanical conversion efficiency, μₐ is the optical absorbance; [symbol] is the optical fluency, and Γ is the thermodynamic conversion coefficient for PA pressure generation, which is also known as the Grüneisen parameter. This formulation, however, is not sufficient to model the PA pressure enhancement due to fluorescence change for the purpose of designing VSD. The presently disclosed formulation starts from the energy conservation rule based on quantum yield; the total amount of absorbed energy by an absorber (i.e., E_{abs}=μₐ [symbol] ) will be converted into thermal energy (Eₜₕ), and light re-emission like fluorescence (E_{F}), and other photochemical reactions (Eₒₜₕₑᵣₛ):

E_{abs}=Eₜₕ+E_{F}+Eₒₜₕₑᵣₛ (3)

and the substitution of Eq. (3) into Eq. (2) gives

p₀=ΓEₜₕ=Γ(E_{abs}−E_{F}−Eₒₜₕₑᵣₛ). (4)

Now, the PA pressure change ratio (i.e., C_{PA}) depending on the neural depolarization can be expressed as

$${C}_{PA} = \frac{{p}_{0}}{{p}_{0}^{′}} = \frac{( {E}_{abs} - {E}_{P} )}{( {E}_{abs}^{′} - {E}_{P}^{′} )} , \qquad ( 5 )$$

where p₀ and p′₀ are the initial PA pressures generated by PAVSD at resting and depolarized states of neurons, assuming that Eₒₜₕₑᵣₛ is negligible compared to E_{F}. E′_{abs} and E′_{F} are the total-energy amount of absorbance and fluorescence emission in action state, respectively. Correspondingly, the total amount of absorbance and fluorescence change depending on the depolarization state of a neuron can be given by

$$\begin{matrix}{C}_{abs} = \frac{{E}_{abs}}{{E}_{abs}^{′}} & ( 6 ) \\ and & \\ {C}_{P} = \frac{{E}_{P}}{{E}_{P}^{′}} . & ( 7 )\end{matrix}$$

In addition, the ratio of fluorescence energy compared to total optical absorbance in the resting state, which is the same as the quantum yield Φ′_{F}, can be given by

$${Φ}_{F}^{′} = \frac{{E}_{P}^{′}}{{E}_{abs}^{′}} = \frac{{C}_{abs}}{{C}_{F}} {Φ}_{F} . \qquad ( 8 )$$

Therefore, the PA pressure change ratio in Eq. (5) can be reformulated as follows:

$${C}_{PA} ≅ \frac{{C}_{P} ( {C}_{abs}^{2} - {Φ}_{F} )}{{C}_{abs} ( {C}_{F} - {C}_{abs} {Φ}_{F} )} = \frac{{C}_{abs} - {Φ}_{F}^{′} \cdot {C}_{P}}{I - {Φ}_{P}^{′}} . \qquad ( 9 )$$

Hence, the PA signal change in response to neuronal depolarization is determined by the combination of the absorbance and fluorescence changes, as well as the ratio of the fluorescence energy and the absorbance energy in the depolarized state.

### B. Representative Photoacoustic Voltage Sensitive Dyes

In some embodiments, the presently disclosed subject matter provides a photoacoustic voltage dependent dye of formula (I):

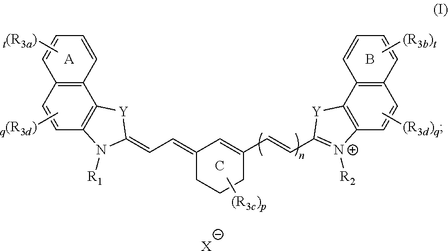

wherein: n is an integer selected from 0, 1, and 2; p and t are each independently integers selected from 1, 2, 3, and 4, q is an integer selected from 1 or 2; A and B can be present or absent; C is a ring that can be present or absent and when present forms part of the polymethine chain, wherein ring C or the polymethine chain of which it forms a part of can be substituted with R_{3c}; R₁ and R₂ can be the same or different and are each independently selected from alkyl, substituted alkyl, and —(CH₂CH₂O)ₘ—R₄, wherein m is an integer from 1 to 20 and R₄ is selected from the group consisting of H, alkyl, and —OR₅, wherein R₅ is alkyl; each R₃ₐ, R_{3b}, R_{3c}, and R_{3d} is selected from the group consisting of alkyl, halogen, hydroxyl, cyano, and alkoxyl; Y is selected from O, S, and C(R₆)₂, wherein each R₆ is independently H or alkyl; X is a counterion; and pharmaceutically acceptable salts thereof. The counter ion can be, for example, Cl⁻, Br⁻, I⁻, OH⁻, and the like.

In some embodiments of the photoacoustic voltage dependent dye, A and B are both present and the dye of formula (I) has the following structure:

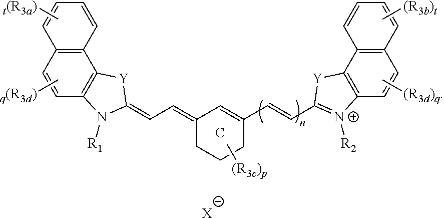

In certain embodiments of the photoacoustic voltage dependent dye, C is absent and the dye of formula (I) has the following structure:

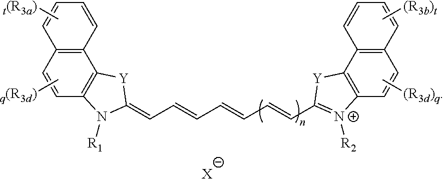

In yet other embodiments of the photoacoustic voltage dependent dye, R₁ and R₂ are each C₁-C₆ alkyl; and Y is C(R₆)₂, wherein each R₆ is C₁-C₆ alkyl. In such embodiments, the dye is:

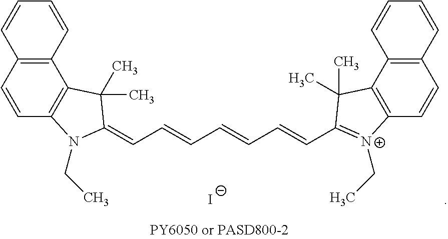 [SMILES: CCN1/C(=C/C=C/C=C/C=C/C2=[N+](CC)c3ccc4ccccc4c3C2(C)C)C(C)(C)c2c1ccc1ccccc21 ; [I-]]

In other embodiments of the photoacoustic voltage dependent dye, R₁ and R₂ are each —(CH₂CH₂O)ₘ—R₄; and Y is C(R₆)₂, wherein each R₆ is C₁-C₆ alkyl. In such embodiments, the dye is selected from the group consisting of:

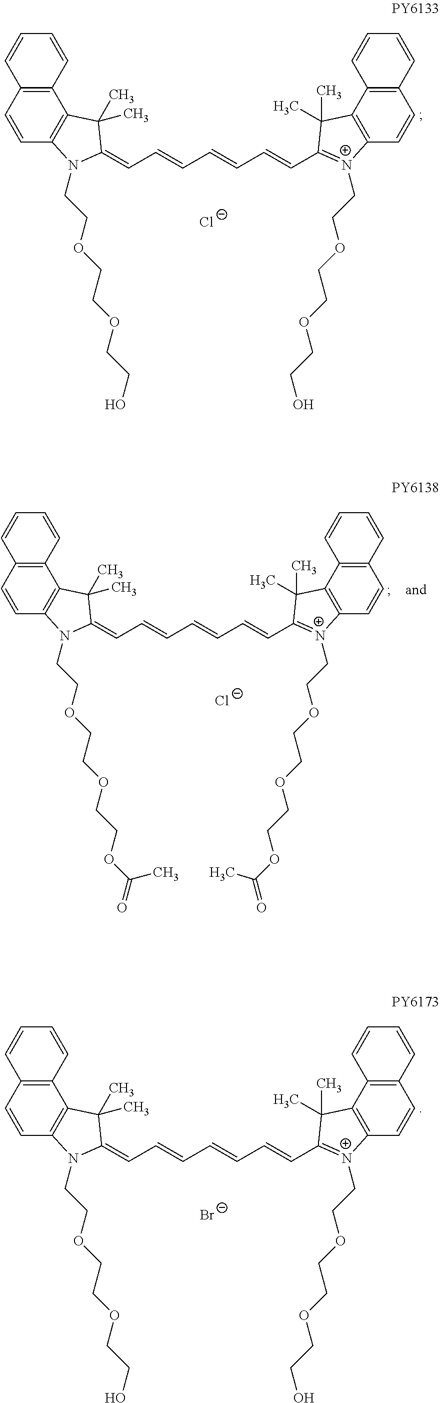 [SMILES: CC1(C)C(/C=C/C=C/C=C/C=C2/N(CCOCCOCCO)c3ccc4ccccc4c3C2(C)C)=[N+](CCOCCOCCO)c2ccc3ccccc3c21 ; [Cl-] ; CC(=O)OCCOCCOCCN1/C(=C/C=C/C=C/C=C/C2=[N+](CCOCCOCCOC(C)=O)c3ccc4ccccc4c3C2(C)C)C(C)(C)c2c1ccc1ccccc21 ; [Cl-] ; CC1(C)C(/C=C/C=C/C=C/C=C2/N(CCOCCOCCO)c3ccc4ccccc4c3C2(C)C)=[N+](CCOCCOCCO)c2ccc3ccccc3c21 ; [Br-]]

In other embodiments of the photoacoustic voltage dependent dye, Y is S. In certain embodiments, the dye is selected from the group consisting of:

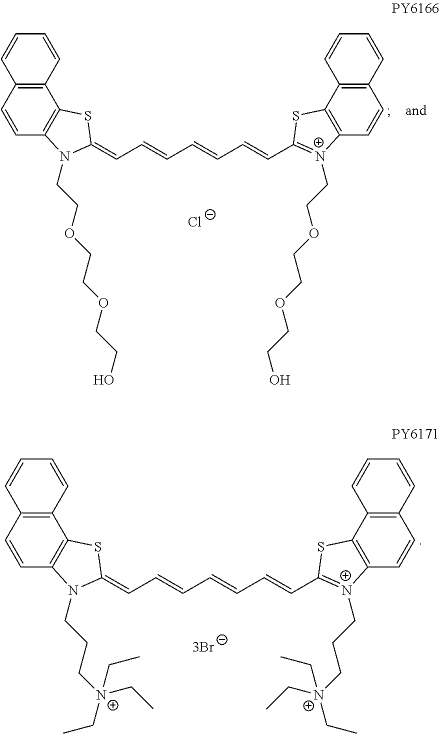 [SMILES: OCCOCCOCCN1/C(=C/C=C/C=C/C=C/c2sc3c4ccccc4ccc3[n+]2CCOCCOCCO)Sc2c1ccc1ccccc21 ; [Cl-] ; CC[N+](CC)(CC)CCCN1/C(=C/C=C/C=C/C=C/c2sc3c4ccccc4ccc3[n+]2CCC[N+](CC)(CC)CC)Sc2c1ccc1ccccc21 ; "3Br"]

In other embodiments of the photoacoustic dye, C is present, A and B are absent, and Y is C(R₆)₂, wherein each R₆ is C₁-C₆ alkyl. In certain embodiments, the dye of formula (I) has the following structure:

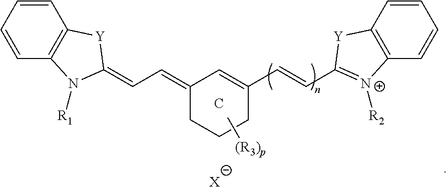

In certain embodiments, the dye is:

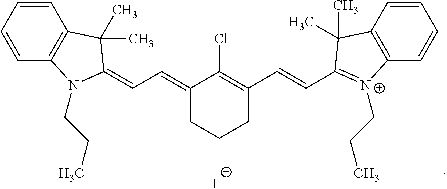 [SMILES: CCCN1/C(=C/C=C2\CCCC(/C=C/C3=[N+](CCC)c4ccccc4C3(C)C)=C2Cl)C(C)(C)c2ccccc21 ; [I-]]

Specifically excluded from the presently disclosed dyes are those dyes disclosed in U.S. Patent Application No. 2015/0073154 A1, to Davis, published Mar. 12, 2015.

In some embodiments, the presently disclosed photoacoustic voltage dependent dyes exhibit a reciprocal mechanism linking fluorescence emission and photoacoustic signals. More particularly, the presently disclosed photoacoustic voltage dependent dyes exhibit the property that upon depolarization, the fluorescence emission is enhanced and the photoacoustic signal is reduced. This relationship is reversed upon repolarization, which provides for the highly specific detection of neuromodulation.

### II. Methods for Imaging

Neuronal depolarization and neurotransmitter release underlie some of the most fundamental components of normal physiology and the etiology of brain pathophysiology. There is a tremendous need for high temporal resolution measurements of neurotransmitter release and its modulation of brain neuronal networks. While there has been progress in measuring neuronal depolarization in vivo in small animals, the current overall methodology of deployment, excitation and measurement of signal from voltage sensitive dyes (VSDs) commonly entails craniotomy and other invasive measures, and thus is currently only practical in animal studies and are not applicable for human studies.

The quantification of neurotransmitter (NT) effects is one of the most fundamental components of the understanding of the underlying workings of the brain. It is the manner in which all neuronal systems communicate with one another by means of synapses. With current methods it is impossible to quantify the effects of NT actions in the cerebral cortex in humans in vivo. The two most abundant NTs, glutamate and GABA, elicit excitation/inhibition of the postsynaptic neurons in one millisecond. In addition, the monoamine transmitters such as dopamine DA, NE, and 5-HT have powerful modulatory network effects in cortical regions of interest, and they are amenable to pharmacological dissection in awake humans, as well as to measures of impact on local networks within defined cortical systems. Currently, neuroscientists measure these effects as membrane potential changes, as well as changes of the number of action potentials with invasive methods (e.g., craniotomy). Methods, such as PET, also are limited in this sense, because they measure binding, but suffer from large time constants (>40 min typically) and poor spatial resolution when the physiological actions of these transmitters are evaluated. In contrast, an imaging method that records NT actions in a spatially defined region in real time would be a great advance. Thus, in some embodiments, the presently disclosed subject matter provides an in vivo brain imaging approach that captures very rapid functional changes of activity induced by NT action with time constants in the millisecond range, using minimally invasive or non-invasive procedures for non-human primates (NHP) and humans.

More particularly, in some embodiments, the presently disclosed subject matter provides photoacoustic detection of neurotransmitter action by delivery of nanosecond pulses to intact skin and skull in response to changed absorption spectra of voltage sensitive dyes. Without wishing to be bound to any one particular theory, it is thought that one also can derive from these voltage depolarizations, regionally active neurotransmitter release, and through pharmacologic manipulation, help derive where the depolarizations have been modulated by neurotransmitters. This will allow understanding of depolarization waves that up to now have not been linked with neuropharmacology directly.

Quantification of changes of photoacoustic signals reflecting changes of neuronal activity and NT action is based on the premise that the absorption spectrum of small molecules changes with neuronal activation. The changes can then be identified and detected by photoacoustics. In the photoacoustic approach, pulses delivered through intact skin and skull are absorbed by the small molecules, i.e., the presently disclosed photoacoustic voltage sensitive dyes, and the generated sound waves can be detected by an ultrasound device. The ultrasound signal is proportional to the absorption of the small molecules delivered to the brain, which changes with neuronal activation in the absorption spectrum. The presently disclosed photoacoustic approach provides realtime neurotransmitter and neurometabolic imaging with greater depth and reduced scatter than current methods known in the art.

One challenge for imaging one or more regions of the brain is the difficulty in crossing the blood-brain barrier with the imaging agent, e.g., a presently disclosed photoacoustic voltage sensitive dyes. Accordingly, the presently disclosed subject matter includes various pathways for delivering the presently disclosed photoacoustic voltage sensitive dyes to the brain. In general, each delivery method has the following characteristics. The solutions can be an isotonic, soluble carrier solution that can be injected into an intravenous vein, and, in some embodiments, for example for injection into humans, sterile and pyrogen free. A typical solution can include a small amount of ethanol or other additives, such as bicarbonate or acidic acid, to stabilize the solution at a physiological pH of approximately 7.4.

One approach for delivery of the presently disclosed photoacoustic voltage sensitive dyes to the brain is direct IV administration as long as the dye is able to travel across the blood-brain barrier. This is the most straightforward approach. Typically, direct intravenous injection requires sufficient lipophilicity, such as a log D of around about 3 to about 4.

In other embodiments, another approach for improving administration of the presently disclosed photoacoustic voltage sensitive dyes to areas of the brain is co-administration of the dye with a second agent, e.g., through pharmacological modulation of adenosine receptor signaling with an agent, such as regadenoson, also known as CVT-3146 or Lexiscan® (Astellas Pharma) or Rapiscan® (GE Heathcare), which can temporarily disrupt the integrity of the blood-brain barrier. A typical dose of regadenoson is 0.4 mg, e.g., 5 mL of 0.08 mg/mL regadenoson administered parenterally.

Nanotechnology approaches also can offer improved retention and controlled release of the presently disclosed photoacoustic voltage sensitive dyes. For example, biodegradable nanoparticles capable of rapidly penetrating the blood-brain barrier are known in the art. For example, densely polyethylene glycol (PEG) coated nanoparticles for a variety of small molecules are known in the art. See, for example, Tang, B. C., Fu, J., Watkins, D. N. and Hanes, J., Enhanced efficacy of local etoposide delivery by poly(ether-anhydride) particles against small cell lung cancer in vivo. Biomaterials 31, 339-44 (2010); Yang, M., Yu, T., Wood, J., Wang, Y. Y. and Tang, B. C., Intraperitoneal delivery of paclitaxel by poly (ether-anhydride) microspheres effectively suppresses tumor growth in a murine metastatic ovarian cancer model. Drug Delivery and Translational Research 1-7, (2014); and Xu, Q., Boylan, N.J., Cai, S., Miao, B., Patel, H. and Hanes, J., Scalable method to produce biodegradable nanoparticles that rapidly penetrate human mucus. J Control Release 170, 279-86 (2013), each of which is incorporated herein by reference in its entirety.

These NPs, when administered intracranially, rapidly distribute within the brain parenchyma. Given the dense PEG coating, these brain-penetrating nanoparticles (BPN) do not adhere to the extracellular matrix as opposed to their non-coated counterparts. Dense PEG coatings on nanoparticles derived from multiple clinically applicable polymers, such as poly(lactic-co-glycolic) acid or poly(sebacic acid) can be adapted to encapsulate small molecules, such as the presently disclosed photoacoustic voltage sensitive dyes. The release kinetics of the dyes can be specifically tailored based on the degradation rates of the nanoparticle system, subsequently allowing the photoacoustic voltage sensitive dyes to interchelate into neuronal membranes and enable voltage measurements.

Another approach for crossing the blood-brain barrier is the use of guided, focused ultrasound, which combines focused ultrasound with magnetic resonance imaging (MRI). Focused ultrasound is a non-invasive delivery method and can be used in conjunction with the nanoparticles described immediately hereinabove. Ultrasound frequencies can range in some embodiments, from about 1 to about 18 MHz, and in more particular embodiments, from about 22 KHz to about 680 KHz. In the presently disclosed methods, the ultrasound frequencies typically range from about a few hundred KHz to about a few MHz range. Commercial systems for performing focused ultrasound include the Insightec Exablate Neuro system (Insightec Ltd., Tirat Carmel, Israel), although any means of producing the desired ultrasound frequencies is within the scope of the presently disclosed subject matter.

Focused ultrasound has been previously demonstrated to be effective in selectively and safely permeabilizing the blood-brain barrier. Accordingly, the presently disclosed photoacoustic voltage sensitive dyes can be loaded into a nanoparticle or a microbubble and delivered across the blood-brain barrier with focused ultrasound to avoid the invasive approach of craniotomy. This approach can be used to efficiently distribute the dyes throughout the brain or to targeted areas of the brain. See also, Sun et al., 2017, who disclose a closed-loop control of targeted ultrasound drug delivery across the blood-brain barrier.

To deliver nanoparticles across the blood-brain barrier using magnetic resonance-guided focused ultrasound, a solution of microbubbles (MBs) and BPN are injected intravenously. Upon exposure to FUS, the MBs oscillate and open the BBB, permitting BPN delivery via convection and diffusion.

Accordingly, the presently disclosed subject matter provides real-time, in vivo photoacoustic imaging and sensing to detect environmentally induced (i.e. due to depolarization) changes in the quantum yield of a dye acoustically, rather than optically. Photoacoustic imaging (PAI) is a hybrid modality that maps the distribution of light absorbing molecules in tissue by virtue of the acoustic waves that they generate when excited by laser pulses. PAI combines the advantageous multispectral and real-time features of optical imaging with the depth and reduced scatter of ultrasound. Real-time spectral imaging is possible at depths several cm including through an intact human skull.

The presently disclosed subject matter extends the field of PAI to the forefront of neuroimaging for in vivo imaging of the human brain and to image NT modulation of the brain network using VSD, enabling sensitive, real-time, deep tissue imaging of brain activity. This requires a combination of dyes with high wavelength (NIR) absorption and sensitivity to neuronal activity, as well as elaborate detector assessment, image generation and mathematical modeling.

In particular embodiments, the presently disclosed subject matter provides a dual-wavelength probe for ratiometric PAI. This approach allows self-calibration for rapid quantitative measurement of neuronal network action. Importantly, this information is in addition to the endogenous PAI contrast (at independent wavelengths) of oxygenation and blood flow.

Either nanosecond laser pulses or RF modulated CW light will be delivered to the intact skin and skull. Once the pulses are absorbed by the dyes in brain tissue, sound waves are generated and detected by an ultrasound transducer-array on the surface of the scalp/skull. This minimally invasive approach will be used to monitor neuronal surface voltage and microenvironmental changes to the absorbance spectra of VSD, respectively.

In addition to applying several light sources (pulsed and modulated CW), the signal-to-noise ratio (SNR) can be determined using different ultrasound receiving configurations including diagnostic linear/phased arrays and sensitive hydrophone. Several beamforming methods can be applied to increase sensitivity and reduce clutter including a short-lag spatial coherency approach. A Phase-locked Loop approach also can be used to maximize the photoacoustic response without increasing the light delivery dosage, using modulated CW laser source. This approach also can enable coded-excitation ultrasound beamforming, which is known to reduce clutter and enhance the image quality.

Accordingly, in some embodiments, the presently disclosed subject matter provides a method for measuring an electrophysiological activity in a subject in vivo, the method comprising administering one or more photoacoustic voltage dependent dyes of formula (I) to a target area of the subject:

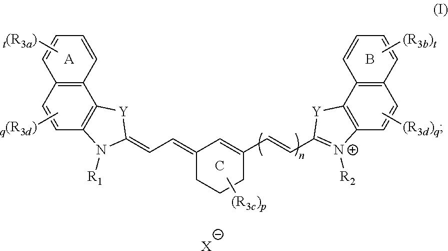

wherein: n is an integer selected from 0, 1, and 2; p and t are each independently integers selected from 1, 2, 3, and 4, q is an integer selected from 1 or 2; A and B can be present or absent; C is a ring that can be present or absent and when present forms part of the polymethine chain, wherein ring C or the polymethine chain of which it forms a part of can be substituted with R_{3c}; R₁ and R₂ can be the same or different and are each independently selected from alkyl, substituted alkyl, and —(CH₂CH₂O)ₘ—R₄, wherein m is an integer from 1 to 20 and R₄ is selected from the group consisting of H, alkyl, and —OR₅, wherein R₅ is alkyl; each R₃ₐ, R_{3b}, R_{3c}, and R_{3d} is selected from the group consisting of alkyl, halogen, hydroxyl, cyano, and alkoxyl; Y is selected from O, S, and C(R₆)₂, wherein each R₆ is independently H or alkyl; X is a counterion; and pharmaceutically acceptable salts thereof. The counter ion can be, for example, Cl⁻, Br⁻, I⁻, OH⁻, and the like.

In particular embodiments, the administering of the one or more photoacoustic voltage dependent dyes of formula (I) includes co-administering the dye with an agent capable of pharmacological modulation of adenosine receptor signaling, incorporating the dye in a brain-penetrating nanoparticle, incorporating the dye in a microbubble, focused ultrasound, and combinations thereof.

In some embodiments, the detecting of the photoacoustic signal comprises ultrasound detection.

In particular embodiments, target area of the subject comprises an organ of the subject. One of ordinary skill in the art would recognize that any organ having membrane conductivity could be imaged by the presently disclosed methods. In yet more particular embodiments, the organ is selected from the group consisting of the brain, heart, kidney, liver, muscle, and the like.

In certain embodiments, the method measures a potential change in an organ, tissue, or cell of the subject.

In further embodiments, the presently disclosed subject matter provides an integrated photoacoustic imaging system for measuring an electrophysiological activity in a subject in vivo, the system comprising:

(a) a near-infrared light source;

(b) an ultrasound probe; and

(c) a data acquisition system.

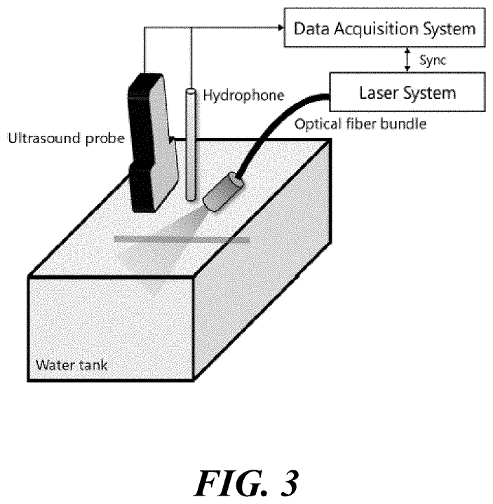

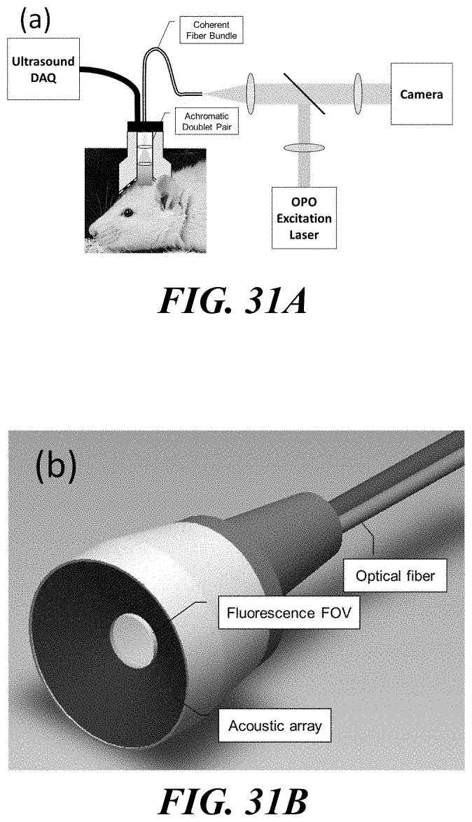

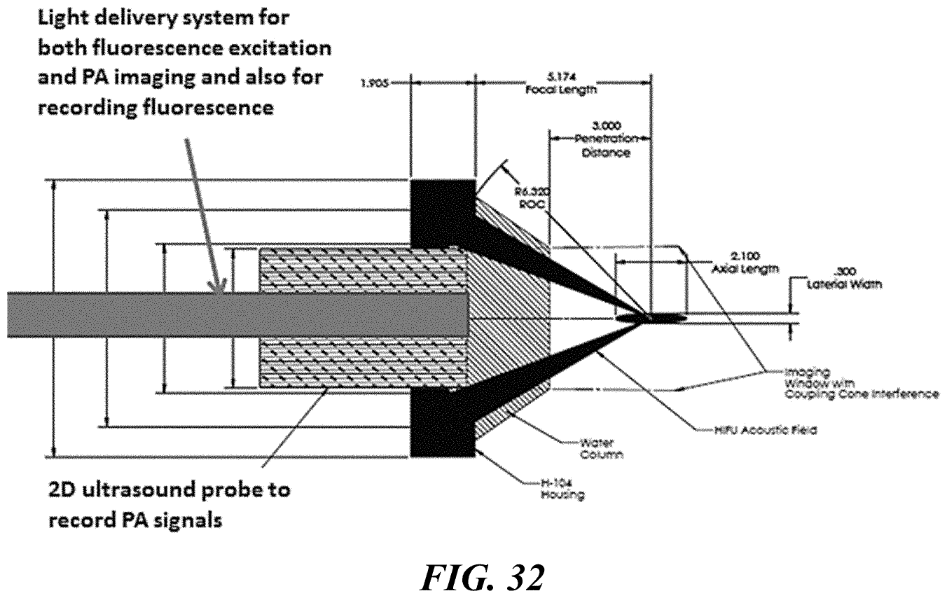

In particular embodiments, the near-infrared light source comprises a tunable laser. Representative imaging systems are provided in FIG. 3, FIG. 31, and FIG. 32. For example, in some embodiments, the imaging system can include a photoacoustic imaging system combined with a fluorescence imaging system, which, in some embodiments, can use a common excitation laser for both photoacoustic and fluorescence excitation. For example, the probe shown in FIG. 31B allows for excitation beam delivery and fluorescence signal collection through a common coherent fiber bundle having, in some embodiments, greater than 100,000 cores. The fiber bundle can be surrounded by an acoustic array for efficient photoacoustic signal collection and beam forming.

In yet other embodiments, the presently disclosed subject matter also provides three-dimensional tomographic reconstruction methods.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, and the like.

### III. Pharmaceutical Compositions and Administration

In another aspect, the present disclosure provides a pharmaceutical composition including a compound of formula (I) alone or in combination with one or more additional therapeutic agents in admixture with a pharmaceutically acceptable excipient. One of skill in the art will recognize that the pharmaceutical compositions include the pharmaceutically acceptable salts of the compounds described above.

In therapeutic and/or diagnostic applications, the compounds of the disclosure can be formulated for a variety of modes of administration, including oral and subcutaneous administration. Techniques and formulations generally may be found in Remington: The Science and Practice of Pharmacy (20^{th} ed.) Lippincott, Williams & Wilkins (2000).

The compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, such agents may be formulated into liquid or solid dosage forms and administered systemically or locally. The agents may be delivered, for example, in a controlled release, timed- or sustained-slow release, or extended release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, e.g., subcutaneous injection, the agents of the disclosure may be formulated and diluted in aqueous solutions, such as in physiologically compatible buffers such as Hank's solution, Ringer's solution, or physiological saline buffer.

The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### IV. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted, for example, with fluorine at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, iso-propyl, cyclopropyl, allyl, vinyl, n-butyl, tert-butyl, ethynyl, cyclohexyl, methoxy, diethylamino, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons). In particular embodiments, the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, iso-pentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a Ci-s alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to Ci-s straight-chain alkyls. In other embodiments, “alkyl” refers, in particular, to Ci-s branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂₅—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)— CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)R′, —C(O)NR′, —NR′R″, —OR′, —SR, and/or —SO₂R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a Ci-20 inclusive straight or branched hydrocarbon moiety having at least one carbon-carbon double bond by the removal of a single hydrogen atom. Alkenyl groups include, for example, ethenyl (i.e., vinyl), propenyl, butenyl, 1-methyl-2-buten-1-yl, pentenyl, hexenyl, octenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “cycloheteroalkenyl” as used herein refers to a saturated monocyclic or bicyclic alkenyl radical in which one carbon atom is replaced with N, O or S. The cycloheteroalkenyl may contain up to four heteroatoms independently selected from N, O or S. Examples of cycloheteroalkenyl groups include, but are not limited to, radicals derived from imidazolyl, pyrazolyl, pyrrolyl, indolyl, pyranyl, and the like. A specific example of cycloheteroalkenyl group is 5-methyl-2-oxo-1,3-dioxol-4-yl.

The term “alkynyl” as used herein refers to a monovalent group derived from a straight or branched Ci-20 hydrocarbon of a designed number of carbon atoms containing at least one carbon-carbon triple bond. Examples of “alkynyl” include ethynyl, 2-propynyl (propargyl), 1-propynyl, pentynyl, hexynyl, heptynyl, and allenyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S— CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxo, arylthioxo, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

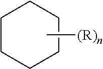

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

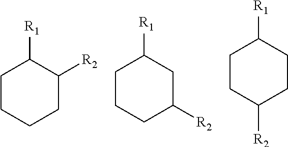

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′— (C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as an acetylfuran and a phenacyl group. Specific examples of acyl groups include acetyl and benzoyl.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, t-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an amide group of the formula —CONH₂.

“Alkylcarbamoyl” refers to a R′RN—CO— group wherein one of R and R′ is hydrogen and the other of R and R′ is alkyl and/or substituted alkyl as previously described. “Dialkylcarbamoyl” refers to a R′RN—CO— group wherein each of R and R′ is independently alkyl and/or substituted alkyl as previously described.

The term carbonyldioxyl, as used herein, refers to a carbonate group of the formula —O—CO—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, iso-propylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —(C═O)— group.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to —SH.

The term ureido refers to a urea group of the formula —NH—CO—NH₂.

Unless otherwise explicitly defined, a “substituent group,” as used herein, includes a functional group selected from one or more of the following moieties, which are defined herein:

(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and

(B) alkyl, heteroalkyl, cycloalkyl, heterocycloalkyl, aryl, and heteroaryl, substituted with at least one substituent selected from:

(i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and

(ii) alkyl, heteroalkyl, cycloalkyl, heterocycloalkyl, aryl, and heteroaryl, substituted with at least one substituent selected from:

(a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and

(b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “lower substituent” or “lower substituent group,” as used herein means a group selected from all of the substituents described hereinabove for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

Throughout the specification and claims, a given chemical formula or name shall encompass all tautomers, congeners, and optical- and stereoisomers, as well as racemic mixtures where such isomers and mixtures exist.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those which are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

As used herein the term “monomer” refers to a molecule that can undergo polymerization, thereby contributing constitutional units to the essential structure of a macromolecule or polymer.

A “polymer” is a molecule of high relative molecule mass, the structure of which essentially comprises the multiple repetition of unit derived from molecules of low relative molecular mass, i.e., a monomer.

As used herein, an “oligomer” includes a few monomer units, for example, in contrast to a polymer that potentially can comprise an unlimited number of monomers. Dimers, trimers, and tetramers are non-limiting examples of oligomers.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The term “pharmaceutically acceptable salts” is meant to include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)— catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

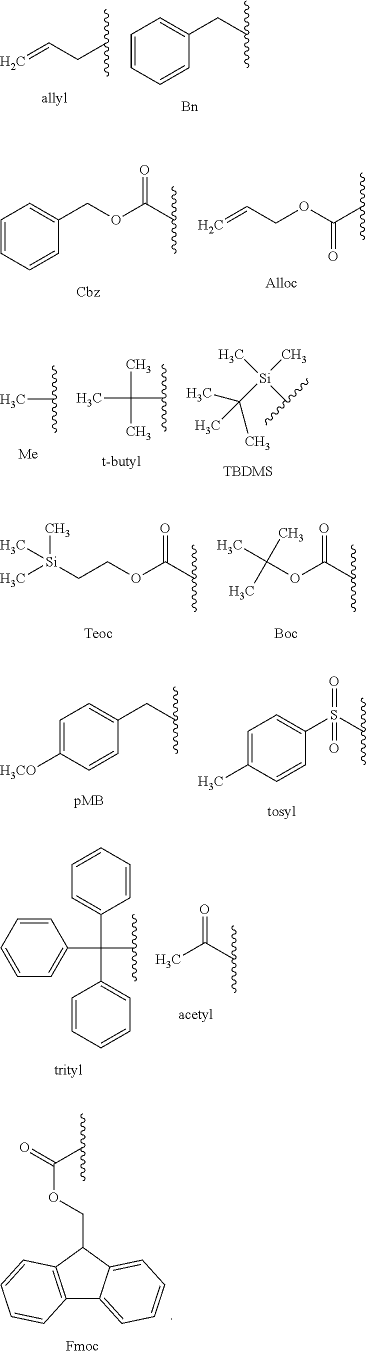

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Photoacoustic Voltage-Sensitive Dyes for In Vivo Imaging

### 1.1 Materials and Methods

### 1.1.1 Synthesis of PAVSD800-2

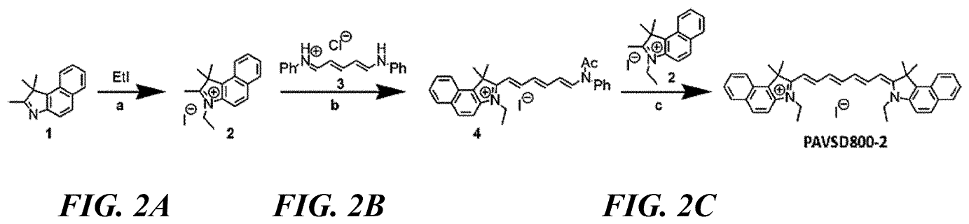

PAVSD800-2 was synthesized by modified literature methods (refer to FIG. 2). (Sinha, et al., 2012; Mujumdar, et al., 1993) 3-Ethyl-1,1,2-trimethyl-1H-benzo[e]indolium iodide (2). (Sinha, et al., 2012) 1,1,2-Trimethyl-1H-benzo[e]indole (1, 1.0 g, 4.78 mmol) and iodoethane (2.8 g, 18.0 mmol) were dissolved in 5-mL acetonitrile under nitrogen. The mixture was sealed in a pressure vessel and allowed to react at 130° C. for 14 h. Upon cooling down to 5° C., the solids formed were filtered out and washed with acetone to give 2 as gray crystals (1.12 g, 64%). ¹H NMR (400 MHz, CD₃OD) δ 1.63 (t, J=7.4 Hz, 3H), 1.84 (s, 6H), 4.68 (q, J=7.4 Hz, 2H), 7.73 (t, J=6.8 Hz, 1H), 7.81 (t, J=6.8 Hz, 1H), 8.02 (d, J=8.8 Hz, 1H), 8.17 (d, J=8.4 Hz, 1H), 8.25 (d, J=9.2 Hz, 1H), 8.33 (d, J=8.4 Hz, 1H); LCMS: m/z=238.0 [M−I]⁺

Benzoindolium 2 (128 mg, 0.35 mmol) and glutaconaldehyde dianil hydrochloride (3, 100 mg, 0.35 mmol) were dissolved in 2 mL of acetic anhydride, and the mixture was allowed to react at 130° C. in a pressure vessel for 1 h. (Mujumdar, et al., 1993) The solution turned purple. The progress of reaction was followed by absorbance spectra in methanol, which showed a major peak at 501 nm upon completion. This reaction mixture was used for the next step directly.

### 1.1.2 Cyanine PAVSD800-2

A second equivalent of benzoindolium 2 (128 mg, 0.35 mmol) and 2 mL of pyridine were slowly added to the intermediate (4) in acetic anhydride. The mixture was allowed to react at 100° C. for 30 min, and the solution turned green. The progress of reaction was followed by absorbance spectra in methanol, which showed a major peak at 780 nm upon completion. After cooling down, the solvents were removed by rotary evaporation and the residue was purified by chromatography (SiO₂-amino bond, 2:98 MeOHCH₂Cl₂). Green colored fractions were combined and the solvents were removed by rotary evaporation. The residue was washed with isopropanol (2×10 mL) and then dissolved in 30 mL 1:2 i-PrOHCH₂Cl₂. The solvents were evaporated slowly under vacuum until about 5 mL was left. Precipitates formed were filtered out and washed with 10 mL i-PrOH, and dried to give cyanine PAVSD800-2 as green powders (94 mg, 40%). R_{f} (silica gel; 1:9 MeOHCH₂Cl₂)=0.64; ¹H NMR (400 MHz, CD₃OD) δ 1.42 (t, J=7.2 Hz, 6H), 1.97 (s, 12H), 4.23 (q, J=7.2 Hz, 4H), 6.32 (d, J=14 Hz, 2H), 6.56 (t, J=12.4 Hz, 2H), 7.46 (t, J=6.8 Hz, 2H), 7.55 (d, J=8.8 Hz, 2H), 7.58-7.68 (m, 3H), 7.95-8.07 (m, 6H), 8.21 (d, J=8.8 Hz, 2H); LCMS: m/z=537.0 [M−I]+

[symbol]

.

### 1.2 Lipid Vesicle Preparation

Lipid vesicles were prepared from 25-mg soybean phosphatidylcholine (type II) suspended in 1 mL of K⁺ buffer, which contains 100 mM K₂SO₄ and 20 mM HEPES. This suspension was vortexed for 10 min and sonicated in bath-type sonicator for 60 min, yielding a translucent vesicle suspension. A Na⁺ buffer containing 100 mM Na₂SO₄ and 20 mM HEPES was prepared. During experiments, 10 μL of vesicle suspension was added to 1 mL of Na⁺ buffer, resulting in an approximately 100:1 K⁺ gradient across vesicle membrane. VSD was added to this suspension. When 2.5 μL of 10 μM valinomycin—a K⁺ specific ionophore—was added, K⁺ ions were transported from inside to outside of vesicle membranes, resulting in a negative membrane potential. This negative potential drives the positively charged VSDs into the vesicles, which causes aggregation of dyes and quenching of fluorescence. Subsequent addition of 2.5 μL of 1 mM gramicidin, a nonspecific monovalent cation ionophore, allows Na⁺ cations to move from outside to inside of vesicle membranes to short circuit the membrane potential.

### 1.3 Experimental Setup for Photoacoustic Voltage-Sensitive Dyes Characterization

### 1.3.1 Spectroscopic/Fluorometric Photoacoustic Voltage-Sensitive Dyes Characterization

A combined spectrophoto/fluorometer system (Spectramax i3x, Molecular Devices) was used to measure both fluorescence and absorbance of the PA-VSD with 1, 3, 6, and 9 μM concentrations. For fluorescence, the measurement was conducted for the spectral range from 750 to 850 nm at 10-nm increment, and the absorbance was measured in the range from 600 to 900 nm at 5-nm increments. Note that 720-nm wavelength was used for excitation during spectrofluorometry of the PA-VSD.

The PA sensing system was employed for the characterization of the synthesized PA-VSD (FIG. 3); Q-switched Nd:YAG laser integrated with an optical parametric oscillator (Phocus Inline and MagicPrism, Opotek Inc.) was used for PA signal generation. With the tunability of the laser system, the spectral responses for near-infrared wavelengths (i.e., 700 to 850 nm at 10-nm interval) were scanned with 20-Hz pulsed repetition frequency and 5-nm pulse duration. The light was delivered through an optical fiber bundle with a 9-mm round shaped output, which was aligned with an ultrasound receiver. The PAVSD in the lipid vesicles suspension was loaded into transparent tubes with 1.27-mm diameter (AAQ04133, Tygon®, SaintGobain Corp.), which were located at around 30 mm depth in the water tank. Note that the temperature of the water was consistently maintained at 22° C. during the experiments. A 10-MHz linear ultrasound probe (L 14-5/38, Ultrasonix Corp.) was used for PA-VSD sensing in a practical circumstance with a limited bandwidth (i.e., 75%-6 dB fractional bandwidth). Similarly, a hydrophone with wide flat bandwidth from 250 kHz to 20 MHz (HGL-1000, Onda Corp., California) was used to collect single line radio-frequency (RF) PA pressure signal. Twenty sets of PA data were collected, in which 19 of them were collected using the linear array transducer, and the other was through the hydrophone, and the average and standard deviation values were calculated. The PA signals acquired at the depth of target were used to form an averaged RF signal, which is further processed to present PA intensity at each optical wavelength; this permitted an accurate correlation with the spectrophotometric measurements. For the imaging setup, one set of data was obtained using the linear array transducer from 9 μM PA-VSD at 800-nm optical wavelength, and the difference between the resting and polarized states was analyzed.

### 1.4 Results

### 1.4.1 Spectrophoto/Fluorometric Photoacoustic Voltage-Sensitive Dyes Characterization

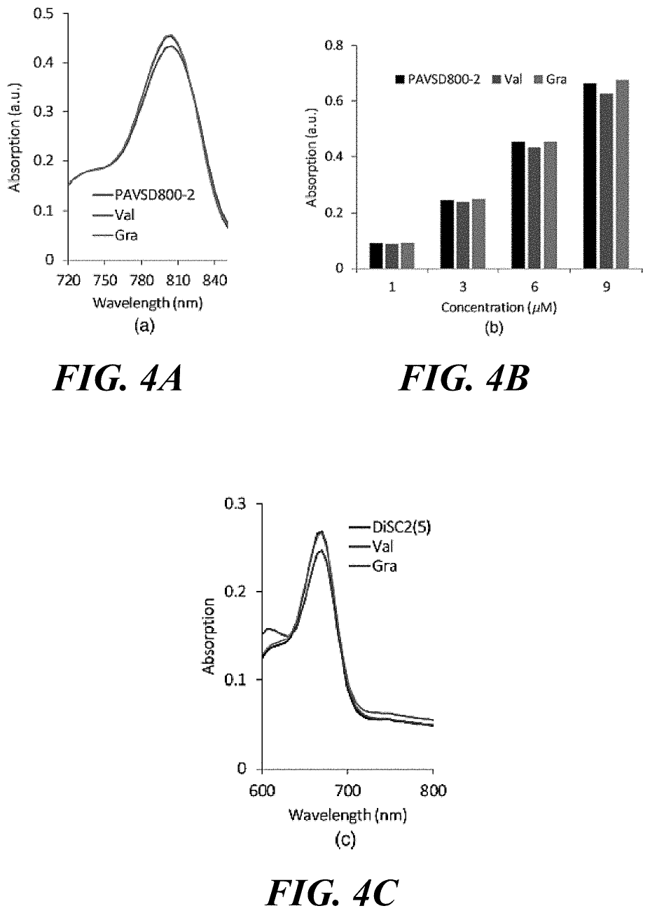

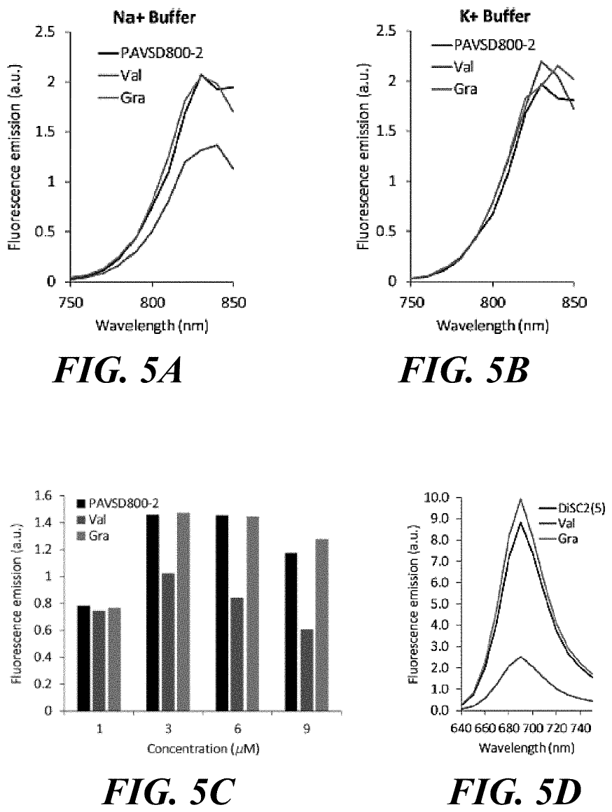

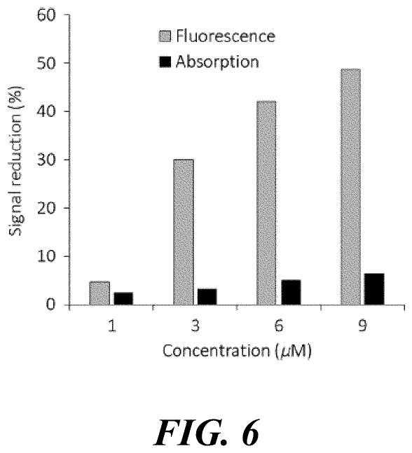

The spectrophoto/fluorometric results of the dye with different concentrations are shown in FIG. 4A, FIG. 4B, FIG. 4C, FIG. 5A, FIG. 5B, FIG. 5C, FIG. 5D, and FIG. 6. In the absorbance results, FIG. 4A shows the absorbance spectrum for PAVSD800-2 at 6 μM concentration, and the peak was determined to be 800 nm. The absorbance slightly decreased in response to the membrane potential at the addition of valinomycin. As the negative control, adding gramicidin into the suspension with valinomycin recovered its absorbance intensity. When the dye concentration of 1, 3, 6, and 9 μM concentrations were tested, a linear trend of absorbance increase was observed by increasing the dye concentration, and a slight reduction during depolarization is seen for all concentrations [FIG. 4B]. A decrease in the primary absorbance peak and an increase in shoulder peak are characteristic of H-aggregation for cyanine dyes. (McArthur, et al., 2012) The measured fluorescence spectra with different PAVSD800-2 concentrations are shown in FIG. 5A, FIG. 5B, and FIG. 5C. The fluorescence quenching effect is due to negative membrane potential generated by the addition of valinomycin, a K⁺ specific ionophore, and subsequent self-quenching of positive dye molecules accumulating and aggregating inside the vesicles. The effect was reversed when gramicidin, a nonspecific ionophore, was added to depolarize the membrane potential. The spectrum change in fluorescence quenching at 6 μM concentration is shown in FIG. 5A. As negative control, K⁺ buffer was used as the external buffer instead of Na⁺ buffer. This control condition establishes the same concentration of K⁺ on both sides of the vesicle membrane, preventing the generation of a polarized voltage when valinomycin is added; the absence of a fluorescence change therefore indicates that the dye does not interact with valinomycin or gramicidin at these concentrations [FIG. 5B] but responds only to membrane electrical potential [FIG. 5A]. The amount of fractional change in fluorescence emission depends on the dye concentration, and the trend at the emission wavelength of 825 nm is shown in FIG. 5C. A fractional change in absorbance and fluorescence emission for different PAVSD800-2 concentrations are compared and summarized in FIG. 6. A small valinomycin-induced absorbance reduction of 2% to 6% were observed from 1 to 9 μM concentrations, while the fluorescence quenching up to 49% was observed for 9 μM concentration. This substantial fluorescence change will contribute to the corresponding PA signal sensitivity to membrane potential. The absorbance and fluorescence spectrum of the classic VSD, di-SC2(5), at the concentration of 6 μM, are also obtained and shown in FIG. 4C and FIG. 5D. This result may be regarded as a positive control to verify that the lipid vesicle system was operating as expected. FIG. 5D particularly serves to highlight that the magnitude of the fluorescence response can be dramatically large and that improved versions of the longer wavelength cyanine dyes may be achievable.

### 1.4.2 Photoacoustic Characterization of the Photoacoustic Voltage-Sensitive Dyes

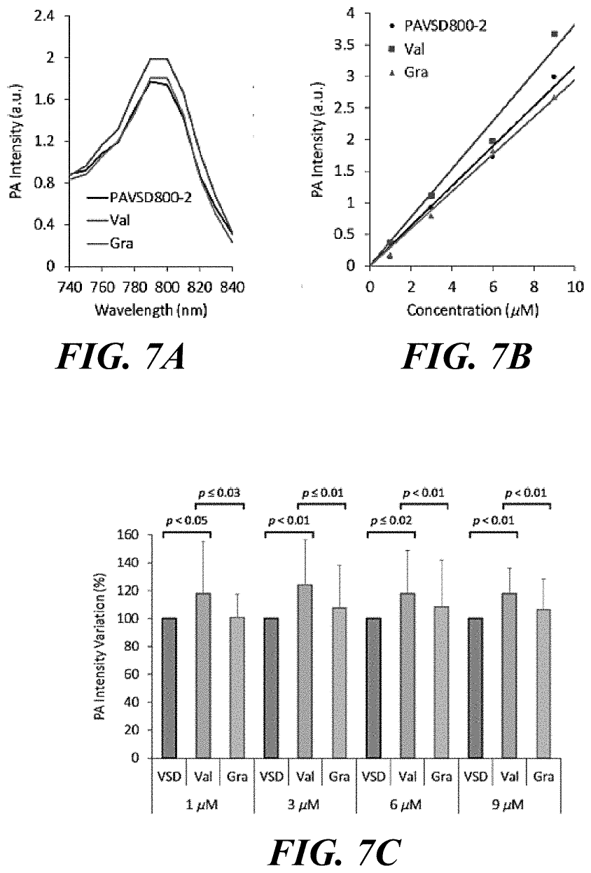

The PA spectrum of 6 μM PAVSD800-2 is shown in FIG. 7A for a typical experiment. Valinomycin administration, which polarizes the lipid vesicle membranes, increases the PA signal intensity. Addition of gramicidin restores the PA signal intensity close to the original level. In this experiment, the PA intensity at 800 nm wavelength increases by 14% in the polarized state. Different concentrations of PAVSD800-2 were also tested at 800 nm [FIG. 7B]. As would be expected, an increase in PA intensity is observed with concentration and the effect of polarization (Val, red) and depolarization (Gra, green) is seen at all concentrations. To confirm the statistical significance of the PA enhancement through polarization, 20 samples were measured, and PA intensity variation was computed by normalizing the intensity to the initial dye intensity. FIG. 7C shows the mean and the standard deviation of PA intensity variation under three conditions (prestimulus, stimulated polarized, and depolarized states). Comparing the initial depolarized state and after adding valinomycin, and the valinomycin result and the result after adding gramicidin, p-values were all below 0.05. The fractional PA intensity changes upon polarization at 1, 3, 6, and 9 μM concentrations were 17%, 24%, 17%, and 17%, respectively.

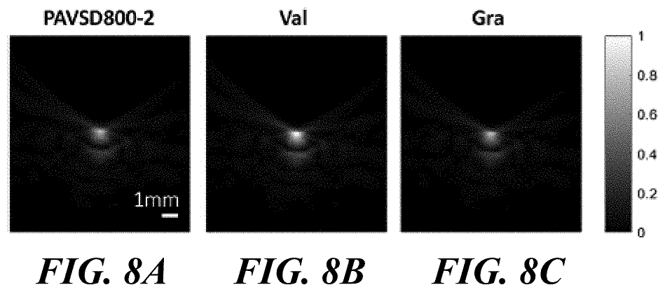

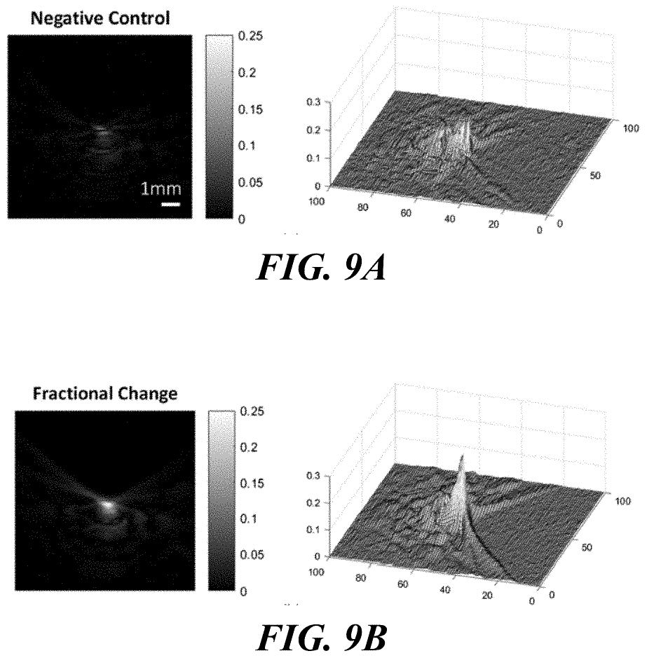

FIG. 8 shows the reconstructed PA images with the 800-nm wavelength. A dataset at 9 μM concentration, which showed 22.72% fractional change of PA signals, was chosen to display. The brighter contrast was observed from the tubing in the valinomycin-induced polarized state, and the contrast was restored to the prestimulus level in the depolarized state after adding the gramicidin. To further emphasize the change in PA intensity, the before and after valinomycin difference images are shown in FIG. 9. The result after adding gramicidin shows a close intensity to the original data, and it is regarded as the negative control of the evaluation. Note that the same color scale was used on these two results, and a clear enhancement in PA signal intensity was seen at the simulated resting state (i.e., the valinomycin induced negative potential) compared to that of the depolarized state.

### 1.5 Discussion

The design of PA-VSDs based on the fluorescence self-quenching mechanism of cyanine VSDs is disclosed. This approach was experimentally validated by designing, synthesizing, and testing a near-infrared absorbing cyanine dye. Based on the results, the use of PA imaging to detect membrane potential events in real-time is demonstrated.

The presently disclosed approach relies on the voltage-dependent mechanism of cyanine dye redistribution through cell membranes. It is important to note that this mechanism can be slow on the timescale of seconds. (Sims, et al., 1974; Loew, et al., 1985; Loew, 2015) This is too slow following the millisecond time scales of neuronal action potentials or electrical events at individual synapses. Studies using two-photon fluorescence imaging microscopy of fast VSDs or genetically encoded voltage indicators can produce high spatial and temporal resolution, <1 ms and <1 μm. (Acker, et al., 2011; Acker, et al., 2016; Walther, et al., 2013; Yan, et al., 2012) Although the cyanine dyes are too slow to capture such single spatially localized events, it is thought that the proposed VSD technology integrated with PA imaging promises to allow deep monitoring of brain activity patterns on the submillimeter spatial scale and second timescale. Spatial resolution in PA imaging mainly depends on the frequency of the ultrasound receiver and the focusing aperture size. For example, when the receiving ultrasound probe has a center frequency of 10 MHz, the maximally expected resolution is 150 μm in theory. Potentially, two-photon excitation could permit localized PA detection down to the micrometer scale. Pronounced electrical events, such as seizure patterns, should be readily imaged.

Additionally, because the slow VSDs effectively integrate electrical activity over space and time, spatiotemporal patterns in response to prolonged stimulus and NT release should also be captured. Of course, the studies presented herein represent the first generation of PA-VSDs with a simple excitation and detection scheme and are intended to demonstrate our design principles. These principles can be used to develop improved PA-VSDs that will capture faster and/or smaller electrical events.

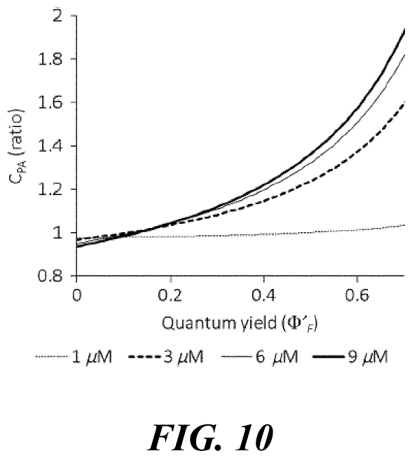

The precise membrane polarization induced by valinomycin cannot be determined for certain in the presently disclosed phantom study, because of some inevitable leakage permeability of the lipid vesicle membranes. Nonetheless, the valinomycin-induced membrane polarization has the upper estimate of −120 mV, which is derived from the 100-fold potassium gradient across the lipid vesicle membranes. From the given polarization, 24% and 49% of fractional changes in PA and fluorescence was achieved at, respectively, 3 and 9 μM PA-VSD. Therefore, the physiological membrane potential changes in the range of −20 to −100 mV should be readily detectable. This sensitivity is considerably higher than the best “fast” VSD used for fluorescence monitoring of excitable cells and tissue. (Yan, et al., 2012) Slow VSD mechanisms can, in general, to produce much higher sensitivities than fast VSDs with ΔF/F as high as 500%/100 mV; (Loew, et al., 1985) it is thought that the sensitivity of PA-VSDs can be improved much further by designing structures with optimized solubility and permeability. Although the measured absorbance reduction and the fluorescence quenching represent two competing processes to control the magnitude of PA sensitivity, the effect of fluorescence quenching was dominant as observed in our experimental results [compare FIG. 4A and FIG. 5A]. In general, measurements of the quantum efficiency Φ_{F} in each membrane state would be needed to be able to make a quantitative prediction of the PA intensity change by applying the theoretical model derived in Eq. (9). Based on this formulation, FIG. 10 shows the PA intensity enhancement for various PAVSD800-2 concentrations over a range of resting Φ′_{F}, where the absorbance and the fluorescence change parameters were taken from the experimental measurement results. This graph demonstrates that the PA intensity change is indeed highly sensitive to the fluorescence quantum efficiency. The quenching effect outweighs the absorption reduction and leads to PA intensity enhancement when the quantum yield is higher than around 0.1. The derived theoretical model in Eq. (9) provides a quantitative tool to guide optimization of PA-VSD performance for potential applications in transcranial PA and fluorescence imaging. This model indicates three directions to design a better fluorescence quenching-based VSD: (1) higher fluorescence quenching effect, (2) less absorbance reduction or larger absorbance enhancement upon aggregation, and (3) higher fluorescence efficiency Φ_{F}. It might be counterintuitive that a higher Φ_{F} would produce a better PA intensity change, but the higher the baseline Φ_{F} is, the greater the opportunity for large quenching becomes, as more energy transfers into nonradioactive decay. Nevertheless, there should be further optimization between fractional contrast change of PA signals and its sensitivity, which may be lowered by the high Φ_{F}. Future work will be focused on enhancing the balanced performance of PA-VSDs guided by the theoretical model. A limitation of the current theoretical model is that the PA or fluorescence change is based on the assumption of a linear system with respect to the energy. However, some studies suggested that the PA intensity could be nonlinearly related to the light energy under certain circumstances. (Chen, et al., 1995; Sarimollaoglu, et al., 2014) Therefore, the theoretical model can be improved by including potential nonlinear sensitivity of the VSD. In addition to the dye features that would improve PA voltage sensitivity, other important dye attributes will also be needed to produce practical PA-VSDs for brain imaging. Such improvement might include: even longer wavelengths to further enhance deep tissue imaging; greater aqueous solubility to allow higher concentrations to be delivered to neuronal tissues; dyes with faster voltage-dependent redistribution kinetics to allow for sensing of activity down to the timescale of action potentials or individual NT events. The presently disclosed subject matter provides both a theoretical and experimental proof of concept to allow PAs to be further developed as a new modality for imaging electrical activity in the brain.

### 1.6 Summary

A PA-based VSD, PAVSD800-2, as a potential tool to monitor membrane potential for brain imaging is provided. The design principle of the dye is to manipulate fluorescence quantum yield to enhance PA intensity through voltage-dependent fluorescence quenching, while the total absorbance remains stable. The theoretical model to predict the PA intensity change based on known photophysical dye characteristics also was derived. This model is experimentally validated, and the reciprocal relationship between PA and fluorescence was demonstrated for different dye concentrations.

### Example 2

### Transcranial Real-Time In Vivo Recording of Electrophysiological Neural Activity in the Rodent Brain with Near-Infrared Photoacoustic Voltage-Sensitive Dye Imaging

### 2.1 Overview

Non-invasive monitoring of electrophysiological neural activities in real-time—that would enable quantification of neural functions without a need for invasive craniotomy and the longer time constants of fMRI and PET—presents a very challenging yet significant task for neuroimaging. The present example presents in vivo proof-of-concept results of transcranial photoacoustic (PA) imaging of chemoconvulsant seizure activity in the rat brain. The framework involves use of a fluorescence quenching-based near-infrared voltage-sensitive dye (VSD) delivered through the blood-brain barrier (BBB), opened by pharmacological modulation of adenosine receptor signaling. Using normalized time-frequency analysis on temporal PA sequences, the neural activity in the seizure group was distinguished from those of the control groups. Electroencephalogram (EEG) recording confirmed the changes of severity and frequency of brain activities, induced by chemoconvulsant seizures of the rat brain. The findings demonstrate that PA imaging of fluorescence quenching-based VSD is a promising tool for in vivo recording of deep brain activities in the rat brain, thus excluding the use of invasive craniotomy.

Non-invasive monitoring of electrophysiological brain activities in real-time (order of milliseconds) is a challenging task of neuroimaging. The presently disclosed approach makes possible quantification of neural functions without need for invasive craniotomy. Existing imaging modalities do not have sufficient transcranial sensitivity with the necessary temporal and spatial resolutions. The present example presents in vivo proof-of-concept results for real-time transcranial photoacoustic imaging of near-infrared voltage-sensitive dye (VSD) signals. The imaging successfully detected the perturbation caused by chemoconvulsant seizures in rat brain. The presently disclosed method is applied to the neuroscientific investigation of rodent brains, with the promise of rapid translation into primate and human brains.

### 2.2 Background

The quantification and monitoring of brain function is a major goal of neuroscience and research into the underlying mechanisms of the working brain. (Friston, 2009; Frost, 2003; Raichle, et al., 2006; Grillner, et al., 2016; Roland, et al., 2014) Toward this objective, several modalities have been introduced for the purpose of appropriate neuroimaging; however, existing methods have limitations. Positron emission tomography (PET) provides high molecular resolution and pharmacological specificity, but suffers from low spatial and temporal resolution. (Vanitha, 2011; Raichle, 1998) Functional magnetic resonance imaging (fMRI) provides higher spatial resolution of brain activity; however, the record is a complex blood-oxygenation level dependent (BOLD) signal with comparatively low temporal resolution and uncertain interpretation. (Logothetis, 2008; Berman, et al., 2006) Optical imaging approaches have been used to monitor the brain function of small animals but have limited dynamic ranges and cover only superficial tissue depths because of light scattering and absorbance during penetration of biological tissue in vivo. (Hillman, 2007; Devor, et al., 2012) The approaches require invasive craniotomy, with problematic long-term consequences such as dural regrowth, greater likelihood of inflammatory cascade initiation, and lack of practicality of translation to non-human primate and ultimately to human studies, including neuropsychiatric disorders. (Heo, et al., 2016) In addition, real-time imaging simultaneously with deep penetration has not been demonstrated. Near infrared spectroscopy (NIRS) non-invasively monitors brain function in real-time (˜1 ms) for deep biological tissues (˜several mm), but suffers from poor spatial resolution (˜1 cm) at those depths. (Strangman, et al., 2013; Torricelli, et al., 2014) Therefore, non-invasive monitoring of electrophysiological brain activities in real-time remains a task at hand in neuroimaging, with the aim to quantify brain functions at high spatial resolution in the depths of brain tissue, without need for invasive craniotomy. To overcome the current challenges, photoacoustic (PA) imaging has been investigated as a promising hybrid modality that provides the molecular contrast of brain function with acoustic transcranial penetration and high spatial resolution. (Wang, et al., 2012; Wang, et al., 2003).

In PA imaging, radio-frequency (RF) acoustic pressure is generated, depending on the thermo-elastic property and light absorbance of a target illuminated by pulsed laser, and it is detected by an ultrasound transducer. Based on this mechanism, several PA approaches to detect electrophysiological brain activities recently have been developed in both tomographic and microscopic imaging modes. Deán-Ben et al. presented in vivo whole brain monitoring of zebrafish using real-time PA tomography of a genetically encoded calcium indicator, GCaMP5G. (Deán-Ben, et al., 2016) Ruo et al. reported PA imaging in vivo of mouse brain responses to electrical stimulation and 4-aminopyridine-induced epileptic seizures by means of hydrophobic anions such as dipicrylamine (DPA). (Rao, et al., 2017) However, these studies used voltage sensing in the visible spectral range (488 nm and 530 nm for GCaMP5G; 500 nm and 570 nm for DPA) that is not suitable for recording of deep neural activity because of the significant optical attenuation by blood.

Provided herein, is a transcranial recording of electrophysiological neural activity in vivo with near-infrared PA voltage-sensitive dye (VSD) imaging during chemoconvulsant seizures in the rat brain with intact scalp. As a step toward non-invasive external imaging in primates and human brains, the results demonstrate that PA imaging of fluorescence quenching-based VSD is a promising approach to the recording deep brain activities in rat brain, without need for craniotomy.

### 2.3 Results

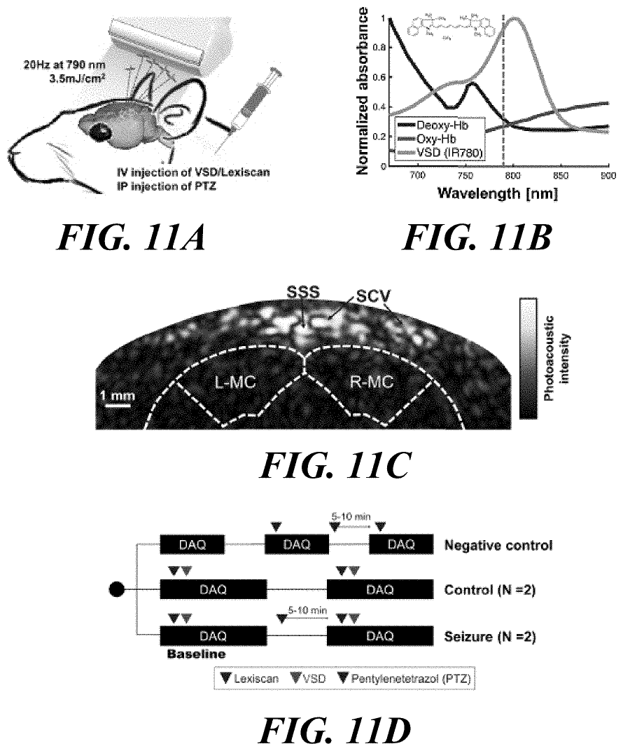

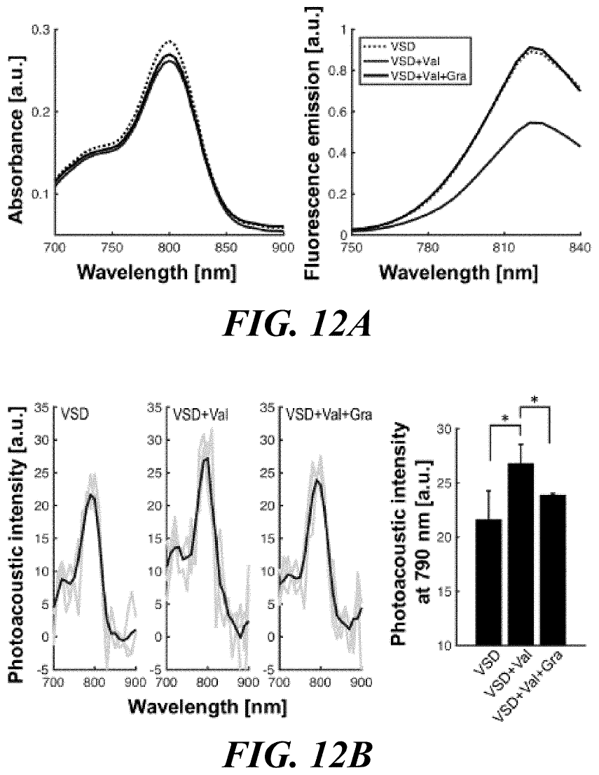

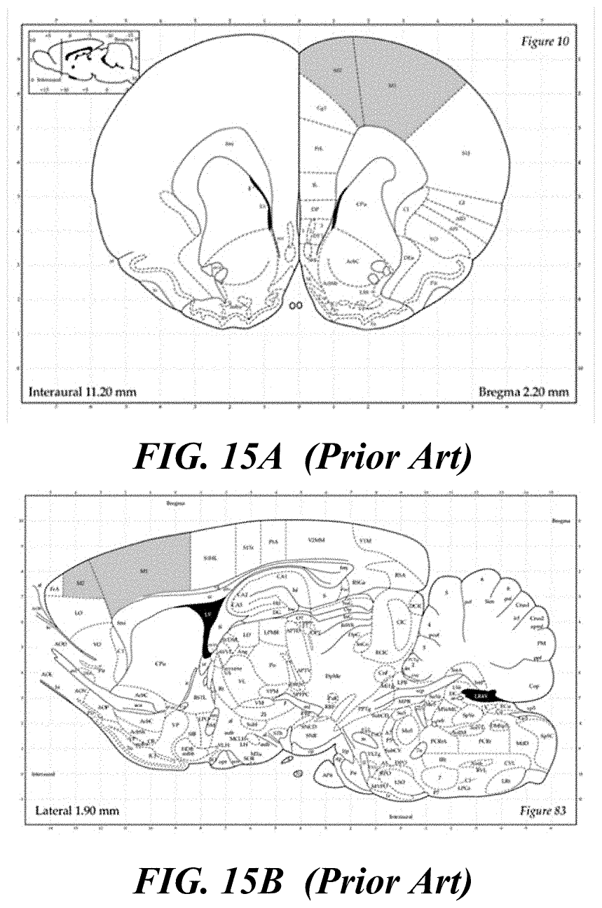

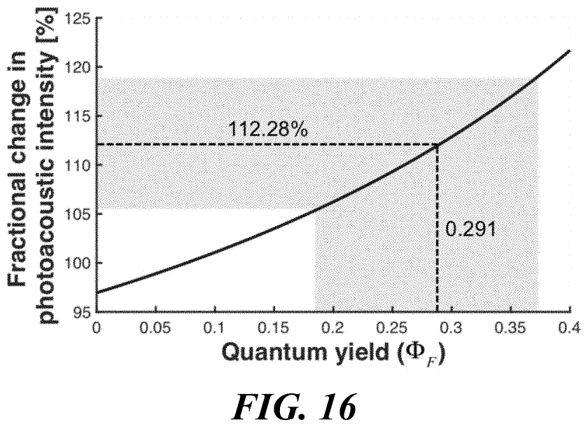

The in vivo PA imaging system was based on a 128-channel array ultrasound transducer and a Nd:YAG laser system with a tunable optical parametric oscillator (OPO) (FIG. 11A). The energy density employed in the experiments was maintained at 3.5 mJ/cm2 that is far below the maximum permissible exposure (MPE) of skin to laser radiation of the ANSI safety standards. (American National Standard for the Safe Use of Lasers, 1975) A wavelength of 790 nm was used, at which the light energy was sufficiently absorbed by the near-infrared VSD, i.e., IR780. Probing at this wavelength avoided the undesired time-variant change of oxygen saturation, being at the isosbestic point of Hb and HbO₂ absorption spectra in the in vivo setup (FIG. 11B). FIG. 11C presents a representative cross-sectional PA image of a rat brain. The outlines for the brain and motor cortex were drawn based on the rat brain atlas (Paxinos, et al., 2014) (FIG. 15). The in vivo experiments were conducted according to the protocol shown in FIG. 11D, with a design composed of seizure, control, and negative control groups. Seizure was not induced in the control group subject to VSD+Lexiscan administration. The negative control group served mainly to monitor the hemodynamic change induced by Lexiscan injection and seizure induction without VSD administration. FIG. 12 demonstrates the presently disclosed in vitro experimental results using a lipid vesicle model. In the spectrophotometric measurements shown in FIG. 12A, 40.1% of fluorescence emission change with IR780 was obtained between polarized and depolarized states, with the administrations of valinomycin and gramicidin, respectively, while having only 3% of corresponding absorbance change. Based on the fluorescence quenching-based VSD mechanism, the PA intensity in the depolarization state decreased by 12.3±6.7% at 790 nm, compared to the polarized state (P=0.044, FIG. 12B). The theoretical model presented in a previous study suggested that the depolarization events of the lipid vesicle changed the quantum yield of VSD by as much as 0.28±0.10 in the depolarized state, from 0.17±0.06 in the polarized state (FIG. 16). (Zhang, et al., 2017) With the confirmation from the in vitro lipid vesicle model, the in vivo validation for transcranial sensing of electrophysiological neural activity in the rat brain was conducted according to the protocol shown in FIG. 11D. The PA probe was located on the cross-section at around the Interaural 11.2 mm, Bregma 2.2 mm to monitor the PA signal change originating from motor cortex (FIG. 15) (Paxinos, et al., 2014), in eight-to-nine-week-old female Sprague Dawley rats anesthetized with ketamine/xylazine. A rat head was stably fixed with stereotaxic equipment, and was shaved for better acoustic/optic coupling.

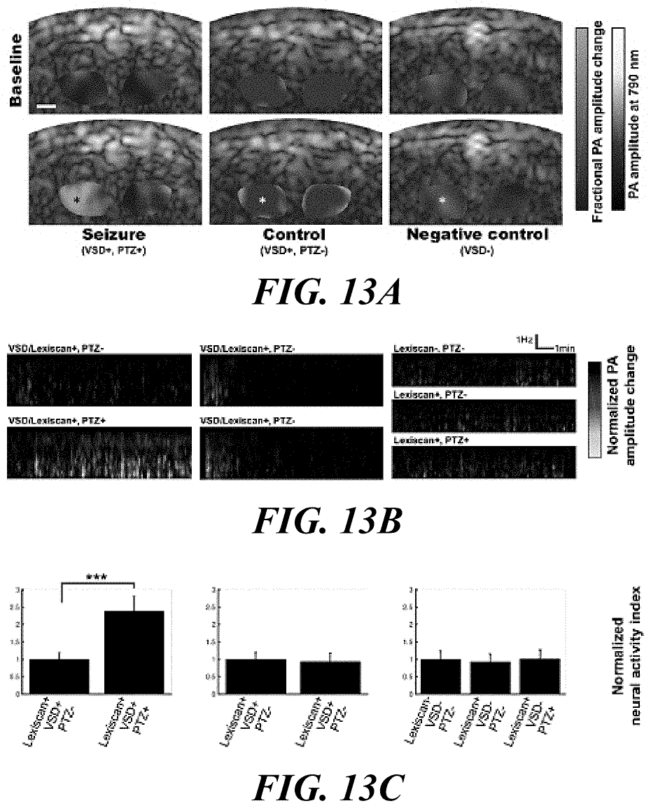

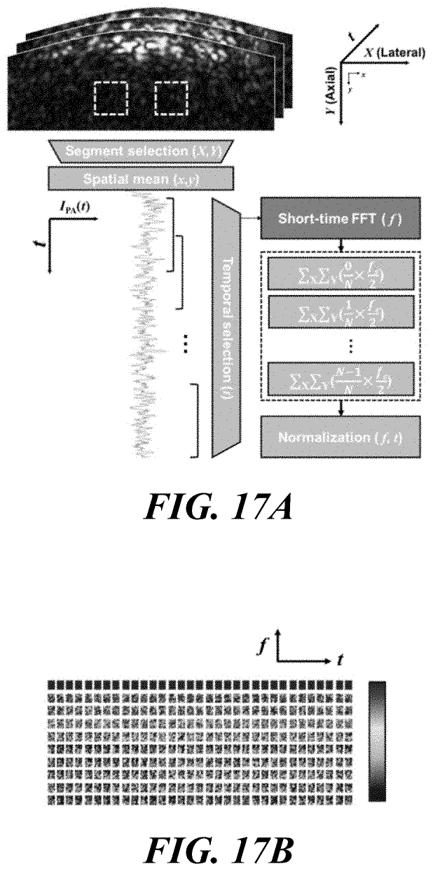

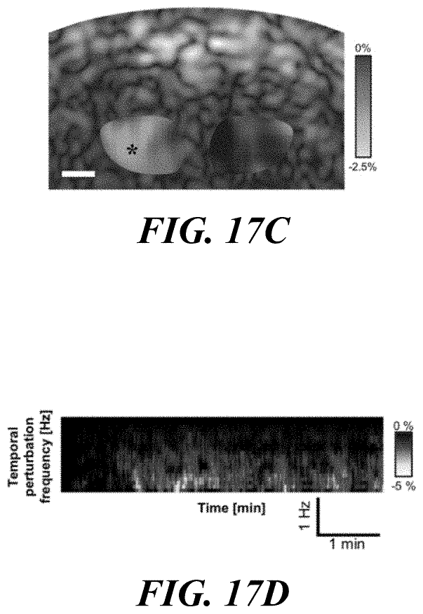

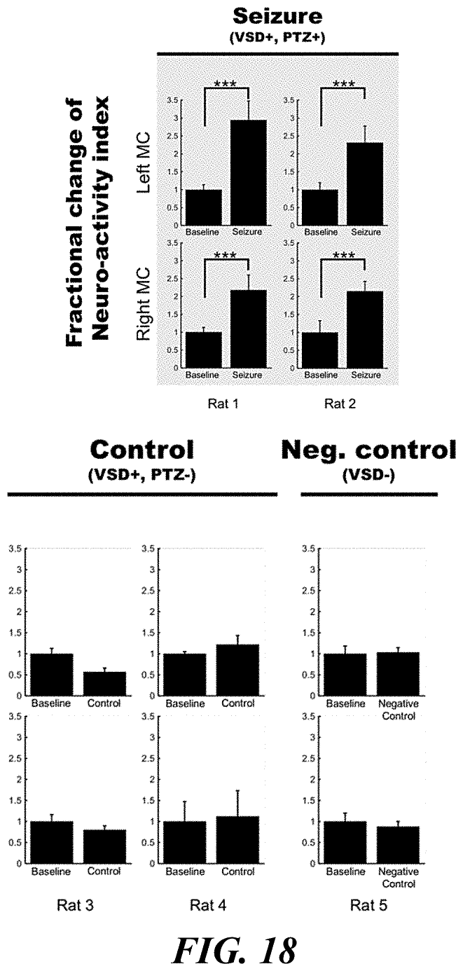

To extract the seizure-induced neural activity from the PA image sequence, the short-time Fourier transform (STFT)-based normalized time frequency analysis method illustrated in FIG. 17 was used. FIG. 13A presents the representative neural activity maps projected in the temporal direction for 10 min in the seizure, control, and negative control groups. The chemoconvulsant seizures induced substantial VSD responses in both motor cortices, while control and negative control groups had consistent records throughout the baseline and comparison phases. The dynamic evolutions of neural activity map over time also were measured (data not shown). In the respective STFT spectrograms obtained from the regions-of-interest (ROIs) indicated by asterisks in FIG. 13A, significant differences between seizure and control groups were also recorded (FIG. 13B). FIG. 13C shows the fractional change of the neural activity index measured from motor cortexes of each experimental group. The seizure group depicted as much as 2.4-fold higher neural activity compared to the baseline: 2.40±0.42 vs. 1.00±0.20 (P<0.0001) that is 11.40 times more than pure VSD contrast, i.e., 12.3%. This higher contrast could be obtained by augmenting the VSD signal component at high frequency, while filtering out the stationary VSD signal components during our normalized time-frequency analysis. Otherwise, the control group indicated consistent neural activity indices compared to those in the baseline phases: 0.93±0.25 vs. 1.00±0.20. The negative control group demonstrated no significant interference in our neural activity sensing: 1.00±0.25, 0.92±0.23, 1.01±0.26 for the cases of Lexiscan−/PTZ−, Lexiscan+/PTZ−, and Lexiscan+/PTZ+, respectively. The neural activity index for each rat is presented in FIG. 18.

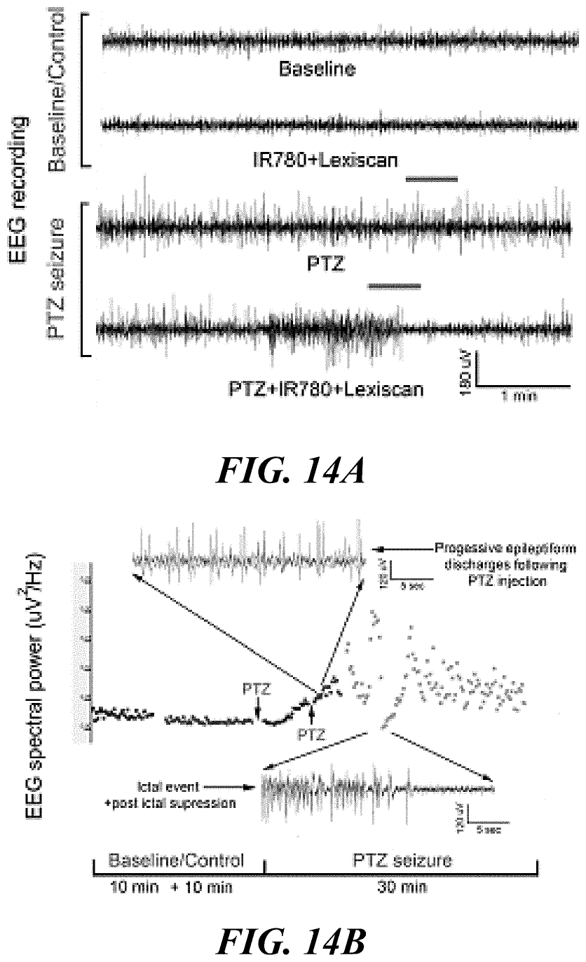

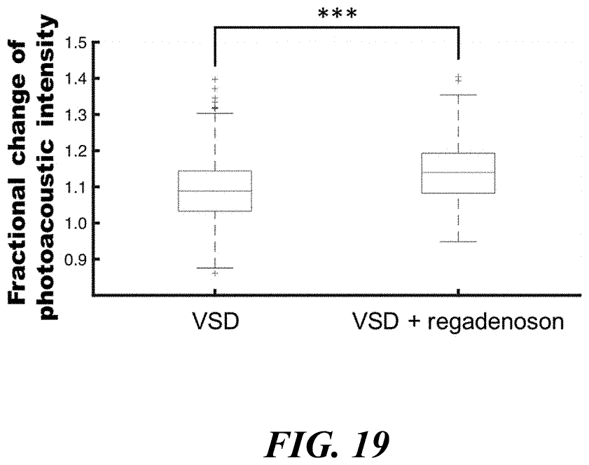

The efficiency of pharmacological treatment for adenosine receptor signaling modulation was evaluated by monitoring the evolution of the PA intensity over time with the intravenous injection of ragadenoson (FIG. 19). The fractional increases from the natural condition (VSD−, Lexiscan−) present statistically significant changes between groups with VSD only and VSD+Lexiscan: 1.09±0.09 and 1.14±0.09, respectively (P<0.0001 with 480 time points) that indicates the VSD penetration into the blood-brain barrier (BBB). The chemoconvulsant-induced seizure activity in the in vivo protocol was validated with EEG recording. Using a well-established model of chemoconvulsant-induced status epilepticus, the classic evolution of chemoconvulsant-induced status epilepticus was replicated using PTZ (FIG. 14). (Loscher, 2017) These evolutions as related to bursts of synchronized neural activity in vivo were assessed in two similar experimental protocols mirrored for the EEG and PA experiments. vEEGs of seizure inductions were recorded using PTZ (45 mg/kg IP injections) in anesthetized rats. EEG baseline recording continued until a stable seizure induction profile (i.e., continuous burst discharges indicating synchronized neuronal depolarization-related action potentials) was recorded using sub-dermal EEG scalp electrodes. The seizure activity in EEG was associated with tonic-clonic movements in the fore- and hind-limbs of the anesthetized rats, indicating motor cortex involvement recorded on synchronous video during EEG acquisition (data not shown). The PTZ evolution of status on EEG did not alter with IV VSD treatment.

### 2.4 Discussion

Here, a transcranial PA recording of electrophysiological neural activity in vivo using near-infrared VSD for chemoconvulsant seizure in rat brain is presented. In the lipid vesicle phantom experiment, the near-infrared VSD, IR780, clearly revealed the signature of the VSD mechanism in polarization/depolarization events induced by valinomycin and gramicidin (FIG. 12). The in vivo validation study demonstrated that the global seizure activity of the motor cortex was clearly differentiated from the activities of the control and negative control groups (FIG. 13). The results also closely agreed with the electrophysiological activities observed by EEG measurement, with an identical experimental setup and protocol (FIG. 14). The potentially confounding factors in the experimental setup and protocol employed need to be carefully considered and eliminated. The change in cerebral blood volume (CBV) during chemoconvulsant seizure can generate fluctuations of PA intensity over time that can be misinterpreted as the suppressive VSD response. (Goldman, et al., 1992; Nehlig, et al., 1996; Hoshi, et al., 1993) To address this concern, two considerations in the in vivo protocol and analysis were adjusted: (1) 5-10 min of the time duration for hemodynamic stabilization were allocated before collecting the PA data, and (2) the STFT spectrogram was normalized in both the frequency and time dimensions. Zhang et al. suggested that the total hemoglobin began to change in the pre-ictal period and remained stable after the initiation of tonic-clonic seizure, and the time length from PTZ injection to seizure onset was ˜2 min on average, (Zhang, et al., 2014) but it was sufficiently covered by our stabilization period in the in vivo protocol. The neural activity map could be stabilized with respect to the CBV change, because the bias on the STFT spectrogram could be rejected during normalization procedures. The negative control group in the in vivo protocol is mainly served to test whether these considerations successfully would work. The PA data obtained without any VSD administration went through an identical analysis method to monitor the chemoconvulsant variation of total hemoglobin concentration and capillary CBV.

As shown in FIG. 13A (right column), comparable neural activity was obtained between the baseline and seizure phases in the negative control group, and there was no significant gradual change of hemodynamics over time, demonstrating that the hemodynamic interferences were successfully rejected. Moreover, instantaneous blood flow perturbation due to heart beating would not affect the results, as every individual PA frame was compounded for two seconds that include 11-16 heart cycles of a rat (typically 5.5-8 beats per second).

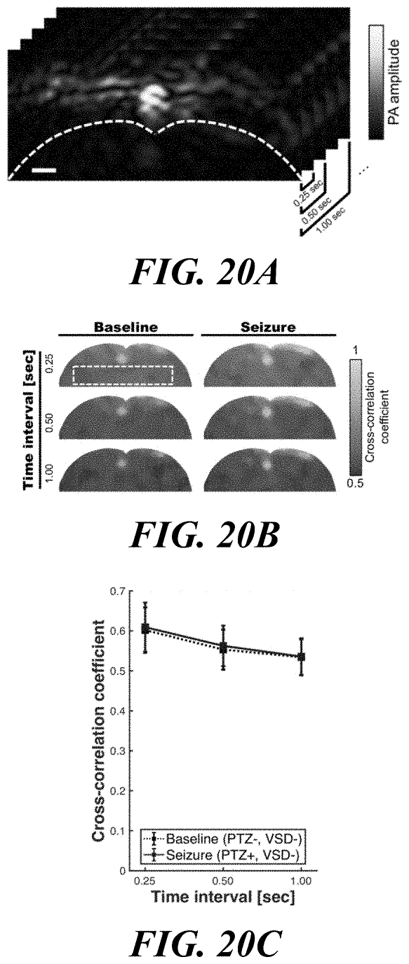

The stability of stereotaxic fixation against the induced motor seizure also was investigated. The counter-hypothesis of this concern was an abrupt disorientation of rat brain due to motor seizure that will induce instantaneous decorrelation between adjacent PA frames. Also, based on the behavioral observation during seizure (data not shown), it was thought that the decorrelation within a sub-second time scale, if it happened. For these hypotheses, the cross correlation maps were calculated throughout PA frames obtained for 8 minutes (1920 frames, 240 frames/min). Three different time intervals were tested: 0.25 sec, 0.5 sec and 1 sec, which respectively correspond to 4, 2 and 1 frame intervals. For each interval, the minimal correlation projection (MCP) map was composed by finding the minimal value per pixel in temporal direction of the entire stack (FIG. 20). The PA frames with seizure indicated no significant decorrelation between adjacent PA frames compared to those obtained without seizure. Therefore, the interference by motor seizure could be rejected as potential cause of artifacts in the results.

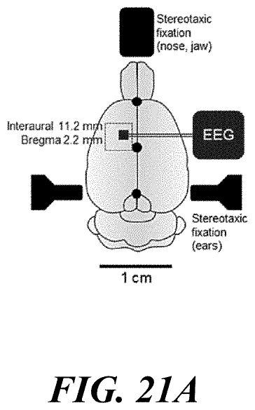

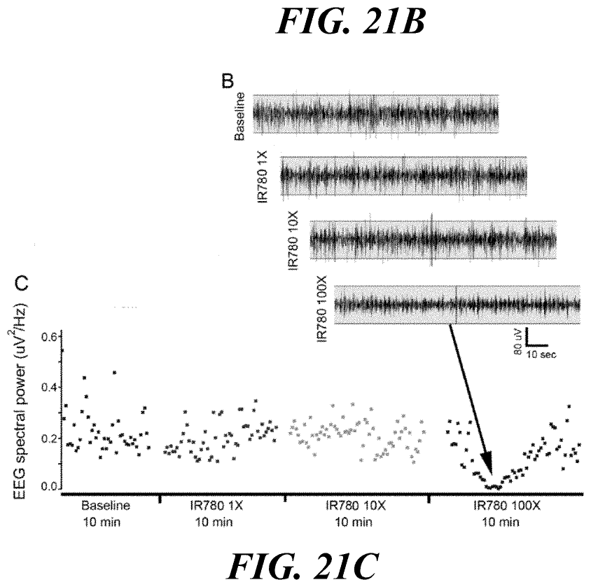

Toxic CNS effects of VSD is another factor that can alter brain activity. The protocols described in FIG. 11D were tested with varying VSD concentration in rats as a direct application to the cortex (FIG. 21). Results for VSD IR780 with cortical application with cranial windows use in six male rats yielded reliable and reproducible EEG signatures using 10-min recordings for each concentration of IR780. This protocol identified that IR780 concentrations had no effect in altering the baseline EEG in the same rat, indicating no toxic effect on cortical circuit function. Direct cortical application with 100× IR780 resulted in significant EEG background suppression in 4/6 rats, indicating that the certain concentrations of VSD could alter baseline circuit function in the motor cortex. This EEG suppression was recovered to baseline over the 10-min recording period, indicating that the transient effect from the time of application as the 100× VSD either diluted or cleared out of the focal application zone over the 10-min period.

Accordingly, the first proof-of-concept of transcranial PA sensing of neural activity with near-infrared VSD, using a chemoconvulsant seizure model of the rat brain, is demonstrated. Other approaches could be used with the presently disclosed methods. For example, the use of localized, non-invasive neural stimulation will allow perspectives in realtime brain response to the external stimuli to be advanced in a totally non-intrusive way. (Lewis, et al., 2016) In particular, it is envisioned that the integration with ultrasound neuromodulation may have a huge impact on the neuroscientific and clinical efforts by enabling the breakthrough beyond the passive brain investigation, while allowing additional benefits on non-pharmacological BBB opening. (Tufail, et al., 2011; Chu, et al., 2015)

Furthermore, the neural sensing speed should be further improved. Current PA sensing speed is limited to 4 frames per second to obtain sufficient signal sensitivity in the deep brain cortex region with the current laser excitation scheme (20 Hz of pulse repetition rate, 3.5 mJ/cm²). This speed may limit its applicability in research, as it is well known that the resting electrophysiological neural activity ranges up to several tens of Hz (e.g., delta: 1-4 Hz; theta: 4-8 Hz; alpha: 8-13 Hz; beta: 13-30 Hz; gamma: 30-50 Hz). (Mantini, et al., 2007) The tradeoff in sensitivity could potentially be resolved by having ˜100 Hz of sensing speed. Successful investigation will substantially increase the capability of the proposed approach for understanding brain function in real-time. In addition, it is thought that improved signal processing for extracting neural activity from the ubiquitous blood context will enable better characterization of brain function. The present in vivo experiments confirmed the possibility of background suppression, but still have artifacts in the sensing area (FIG. 13A). Enhanced signal processing and/or use of multi-spectral wavelengths may allow significantly improved spectral unmixing of electrophysiological activities in the brain, leading to development of novel quantitative metrics for real-time brain characterization.

### 2.5 Methods

2.5.1 Fluorescence Quenching-Based Near-Infrared Voltage-Sensitive Dye. Several cyanine VSDs have been proposed as markers for real-time electrical signal detection (Treger, et al., 2014), and applied for optical imaging of the mitochondrial membrane potential in tumors (Onoe, et al., 2014) and fluorescence tracking of electrical signal propagation on a heart. (Martišiené, et al., 2016) Recently the mechanism of action of a cyanine VSD on the lipid vesicle model was presented. (Zhang, et al., 2017) The discussed mechanism of VSD proposes a suppressive PA contrast when neuronal depolarization occurs, while yielding an enhancing contrast for fluorescence. In the present proof-of-principle study, the fluorescence quenching-based near-infrared cyanine VSD, IR780 perchlorate was used (576409, Sigma-Aldrich Co. LLC, MO, United States) with the analogous chemical structure of PAVSD800-2 in a previous study. (Zhang, et al., 2017) This VSD yields fluorescence emission leading to a reciprocal PA contrast with non-radiative relaxation of absorbed energy.

2.5.1 Photoacoustic imaging setup. For the recording of electrophysiological brain activities in vivo, an ultrasound research system was utilized that consisted of ultrasound array transducer (L 14-5/38) connected to a real-time data acquisition system (SonixDAQ, Ultrasonix Medical Corp., Canada). To induce the PA signals, pulsed laser light was generated by a second-harmonic (532 nm) Nd:YAG laser pumping an optical parametric oscillator (OPO) system (Phocus Inline, Opotek Inc., USA). The tunable range of the laser system was 690-900 nm and the maximum pulse repetition frequency was 20 Hz. The laser pulse was delivered into the probe through bifurcated fiber optic bundles, each 40 mm long and 0.88 mm wide. The PA probe was located at around the Interaural 11.2 mm and Bregma 2.2 mm to obtain the cross-section of motor cortexes (FIG. 15). The energy density at skin surface was only 3.5 mJ/cm², which is far below the ANSI safety limit restricting the 5-ns pulsed laser exposure at 700-1400 nm of wavelength range to be lower than 20 mJ/cm². 2.5.2 Lipid vesicle phantom preparation for VSD validation. The lipid vesicle model was prepared using the same procedure as in Zhang et al (Zhang, et al., 2017); 25-mg soybean phosphatidyl-choline (type II) suspended in 1 mL of K⁺ buffer was used as the lipid vesicles. This vesicle contains 100 mM K₂SO₄ and 20 mM HEPES. The suspension was vortexed for 10 min, and followed by 60 min of sonication within bath-type sonicator to yield a translucent vesicle suspension. A Na⁺ buffer was also prepared, which contains 100 mM Na₂SO₄ and 20 mM HEPES. Afterwards, approximately a 100:1 K⁺ gradient across vesicle membrane was established with 10 μL of lipid vesicle suspension added to 1 mL of Na⁺ buffer. In the vesicle phantom prepared, negative membrane potential (polarized state) was mimicked by adding 2.5 μL of 10 μM valinomycin—a K⁺ specific ionophore, thereby K⁺ ions were transported from inside to outside of vesicle membranes.

Otherwise, 2.5 μL of 1 mM gramicidin, a nonspecific monovalent cation ionophore, enables Na+ cations to move from outside to inside of vesicle membranes to short circuit the membrane potential (depolarized state). From these controls, our near-infrared VSD positively charged can move in and out through the vesicle membrane, leading to the change in fluorescence quenching depending on their aggregation status.

2.5.3 Estimation of quantum yield change of VSD. The quantum yields of the near-infrared VSD in depolarized states (4′F) were estimated based on the Eq. 8 and 9 disclosed previously. (Zhang, et al., 2017) The ratio of absorbance and fluorescence emission in depolarized states (C_{abs} and C_{F}) were 0.97 and 0.60 compared to those in polarized states, respectively, and the estimated fractional changes of PA intensity were calculated for test quantum yields varying from 0 to 0.4 with 0.001 intervals. From the results, the optimal Φ′_{F} was chosen, for which the fractional change of PA intensity obtained in lipid vesicle phantom study was presented. The quantum yield in the polarized state (Φ_{F}) were also estimated by compensating for the absorbance and fluorescence emission changes when depolarized: Φ_{F}=(C_{F}/C_{abs})Φ′_{F}.

2.5.4 Animal preparation. For the proposed in vivo experiments. 8-9-week-old male Sprague Dawley rats weighing 275-390 g were used (Charles Rivers Laboratory, Inc., MA, United States). The use of animals for the proposed experimental protocol was approved by the Institutional Research Board Committee of Johns Hopkins Medical Institute (RA16M225). All animals were anesthetized by intraperitoneal injection with a ketamine (100 mg/ml)/xylazine (20 mg/ml) cocktail. (3:1 ratio based on body weight at 1 ml/kg). The hair was shaved from the scalp of each rat for improved optical/acoustic coupling for transcranial PA recording. The head of the anesthetized rat was fixed to a stable position using a standard stereotaxic device. This fixation procedure was required to prevent any unpredictable movement during PA recording of neural activities.

2.5.5 Chemoconvulsant seizure induction. Penetylenetetrazole (PTZ), a gamma-aminobutyric acid (GABA) A receptor antagonist was used to induce acute seizures in the animals. (Löscher, 2017) To induce global acute seizure in rat brain, an intraperitoneal (IP) injection of PTZ (45 mg/ml) was utilized based on the animal's body weight in a volume of 1 ml/kg. Subsequent doses were given if no acute motor seizure was observed in 5-10 minutes after the first PTZ injection. Generally, 1-2 doses were sufficient to induce the motor seizures in our experiments.

2.5.6 Pharmacological treatment for VSD delivery into blood-brain barrier. The lumen of the brain microvasculature consists of brain endothelial cells, and the blood-brain barrier (BBB) is comprised of their tight junctions to control the chemical exchange between neural cells and cerebral nervous system (CNS). In this study, the penetration through BBB were achieved with a pharmacological method using FDA-approved regadenoson (Lexiscan, Astellas Pharma US, Inc. IL, United States). This modulates the Adenosine receptor signaling at BBB layer. (Carman, et al., 2011) For preliminary studies, the dosage and IV administration method indicated by the manufacturer was utilized. A volume of 150 μl of the standard concentration of 0.08 mg/1 ml was given to each animal regardless of the weight, followed by 150 μl flush of 0.9% sodium chloride for injection. VSD penetration was expected during the Lexiscan's biological half-life, i.e., 2-4 minutes, thereby the experimental protocol was designed based on the pharmacological assumption. In vivo experimental protocol. The in vivo protocols were respectively designed for three experimental groups: negative control, control, and seizure groups. FIG. 11D shows the detailed protocol for each group to present the response to the administration of IR780, Lexiscan, and PTZ. Note that each data acquisition was performed for 10 min to cover the biological half-life of Lexiscan (2-3 min). Each dosing protocol of Lexiscan and VSD was as follows: through the jugular vein catheter port located in the neck, 150 μl of Lexiscan 0.4 mg/5 ml concentration was injected, and 300 μl of VSD was subsequently administrated at 0.1 mg/ml concentration, followed by 150 μl of saline solution flush. The control and seizure groups were designed to distinguish the chemoconvulsant effects on neural activity: both groups received IR780 and Lexiscan, but only seizure group had intraperitoneal (IP) injection of PTZ (45 mg/ml/kg). The induction of seizure was confirmed by monitoring motor seizure, and another dose of PTZ was injected intraperitoneally when no motor seizure was observed in 5-10 min. The negative control group was designed to validate the incapability of Lexiscan and PTZ to generate any bias on PA neural activity detection by excluding VSD administration. In the negative control group, first data acquisition was conducted with the Lexiscan dosage, and the second data set was obtained during the chemoconvulsant seizure with Lexiscan dosage.

2.5.7 Criteria for selecting region-of-interest for STFT spectrogram. The regions-of-interest (ROI) were selected from left and right motor cortices in a PA image. The detailed criteria to select the appropriate ROI were as follows: (1) The minimal size of the ROIs on each hemisphere was set to 1.86×1.54 mm² included within the motor cortex region whose overall dimension is approximately 3×2.5 mm² based on the anatomy of the rat brain atlas shown in FIG. 15. (Paxinos, et al., 2014) The positions of ROIs were at least 2.75-mm below the scalp surface to avoid the regions of several other layers covering a brain, i.e., periosteum, skull, dura mater, arachnoid, subarachnoid space, and pia mater. (Nowak, et al., 2011) Following this criterion also allowed circumventing the dominant clutter signals from large vessels such as superior sagittal sinus (SSS) and superior cortical veins (SCV) nearby the dura mater, thereby the interference on neural activity response in PA signal was prevented.

2.5.8 Normalized time-frequency analysis. The real-time video of suppressive PA variation on motor cortex was reconstructed by expanding the ROI to cover the entire brain tissue region, and computing the localized STFT spectrogram for its segments (5×5 pixels, 18.6×19.3 μm² in dimension). The individual temporal frequency components of the STFT spectrograms were projected in the frequency domain to indicate the total amount of suppressive PA variation. To analyze this non-stationary PA intensity series in the time domain, the temporal analysis window was selected with 2-sec of time duration (40 samples) at the 0.5-sec interval, which enables the temporal frequency analysis up to 2 Hz with the refreshing rate at 4 Hz. FIG. 17 demonstrates the flow chart of our normalized time-frequency analysis to reconstruct the each STFT spectrogram representing PA fluctuation at different frequency. The processing consists of 4 steps for each segment as following:

1. Step 1: short-time Fourier transform of a segment;

2. Step 2: frequency normalization by the baseband intensity f₀ (i.e., 0.1 Hz): PA(t, f)=PA(t, f)/PA(t, f₀), where PA(t, f) and PA(t, f) are the PA sequence before and after temporal normalization;

3. Step 3: linear weighting of each temporal frequency component (i.e., 0.05 to 1 at 0.05 interval for 0 to 2 Hz temporal frequency component at 0.1 Hz interval);

4. Step 4: temporal normalization to obtain PA(t, f)=|PA(t, f)/PA(t, f₀)|, in which the PA₀(t, f) was the averaged intensity during first 1 min after the VSD injection at each frequency component, f;

5. Step 5: Construction of the dynamic neural activity maps by allocating the averaged value of PA(t, f). in the frequency dimension at each time point.

Note that the linear weighting of each frequency component at Step 2 reflected the assumption that VSD responses fluctuating at higher frequency component indicates more vigorous neural activity, while signal component at lower frequency represent the consistent PA signal from blood context plus stationary VSD response in polarized state. Therefore, this procedure yields higher contrast resolution than pure VSD contrast between polarized and depolarized states (i.e., 12.22%, FIG. 12B) since it augments the fluctuating VSD responses, while suppressing the stationary signals. To derive the neural activity index of each rat shown in FIG. 13B, 20 measurements for each rat have employed by dividing the temporal sequences of PA(t, f) in every 1 min duration (240 frames) for left/right motor cortexes to calculate the mean values and their standard variations.

2.5.9 EEG validation of neural seizure activity. To obtain the EEG records of electrical spike discharges that originated from motor cortex shown in FIG. 14, sub-dermal scalp EEG recording electrodes were located at the corresponding locations on motor cortex indicated by Xs in FIG. 21A, the schematic of the rat cranium (three electrodes, 1 recording and 1 reference over motor cortex, 1 ground electrode over rostrum). The EEG signal at motor cortex was recorded with the identical preparation procedures in PA imaging including animal preparation, administration of IR780, Lexiscan, and PTZ, time duration for recording, and interval between sequences in the protocol. Data acquisition was done using Sirenia software (Pinnacle Technologies Inc., Kansas, USA) with synchronous video capture. Briefly, the data acquisition and conditioning system had a 14-bit resolution, sampling rates of 400 Hz, high pass filters of 0.5 Hz and low pass filters of 60 Hz. The files were stored in .EDF format and scored manually for protocol stages using real time annotations added to the recording during the experiments. EEG power for 10 sec epoch displays within the scoring software package was done using an automated module in Sirenia. Further details of our proposed EEG data acquisition and analysis used in this study are as presented in previous studies. (Adler, et al., 2014; Johnston, et al., 2014)

2.5.10 Validation of protocol for using sub-dermal EEG electrodes to determine toxic CNS effects of VSDs. VSDs effect on EEG signal from sub-dermal scalp electrodes was investigated in a pilot by direct cortical applications of increasing concentrations of VSDs in anesthetized rat with limited craniotomies while recording synchronous vEEGs (FIG. 21).

Increasing concentrations of VSD were tested in the same rat at temporally spaced time-points. Rats were anesthetized with IP injection to ketamine/xylazine and a cranial window made over the right motor cortex. After recording a baseline EEG in the rat for 10-min duration with the craniotomy, the follow-on EEG recording continued to record EEG following application of increasing concentrations of vehicle alone and VSD+ vehicle for the same duration of EEG recordings (i.e., 10 min) allowing comparisons of EEG responses to each increasing gradient of VSD on cortical activity as compared to baseline EEG signature in the same rat.

### Example 3

### Correlation of Photoacoustic-Based Neurotransmitter Sensing with Microdialysis

3.1 Materials and Methods

3.1.1 Animal preparation. For the in vivo experiments, 8-9-week-old male Sprague Dawley rats weighing 275-390 g were used (Charles Rivers Laboratory, Inc., MA, United States). The use of animals for the proposed experimental protocol was approved by the Institutional Research Board Committee of Johns Hopkins Medical Institute (RA16M225). The hair was shaved from the scalp of each rat for improved optical/acoustic coupling for transcranial PA recording. The head of the anesthetized rat was fixed to a stable position using a standard stereotaxic device. This fixation procedure was required to prevent any unpredictable movement during PA recording of neural activities.

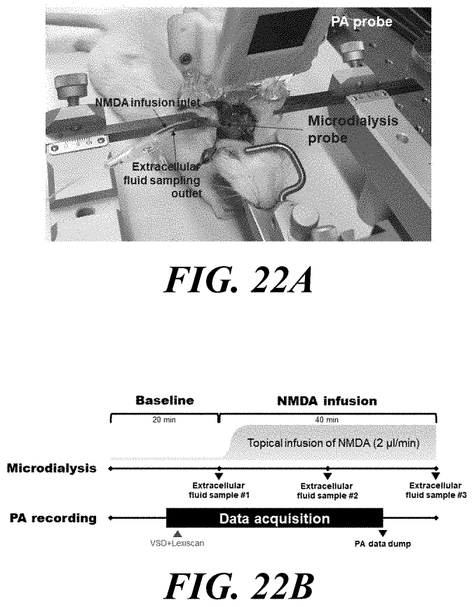

3.1.2 Microdialysis setup. The microdialysis probe consists of a shaft with a semipermeable hollow membrane tip, which is connected to inlet and outlet tubing. The extracellular fluid samples were continuously acquired before and after the infusion of 0.3-mM NMDA through the inlet of the microdialysis probe (FIG. 22A). Each acquisition of extracellular fluid sample took 20 min with 2 μL/min flow rate. From the samples, the change of glutamate concentration was measured.

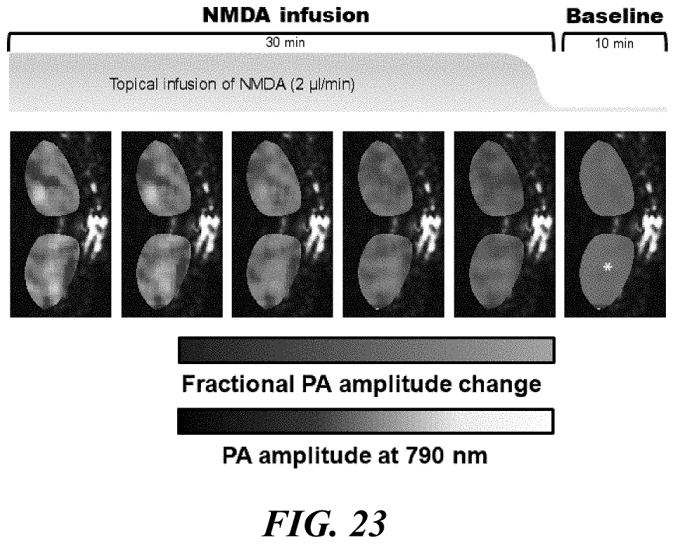

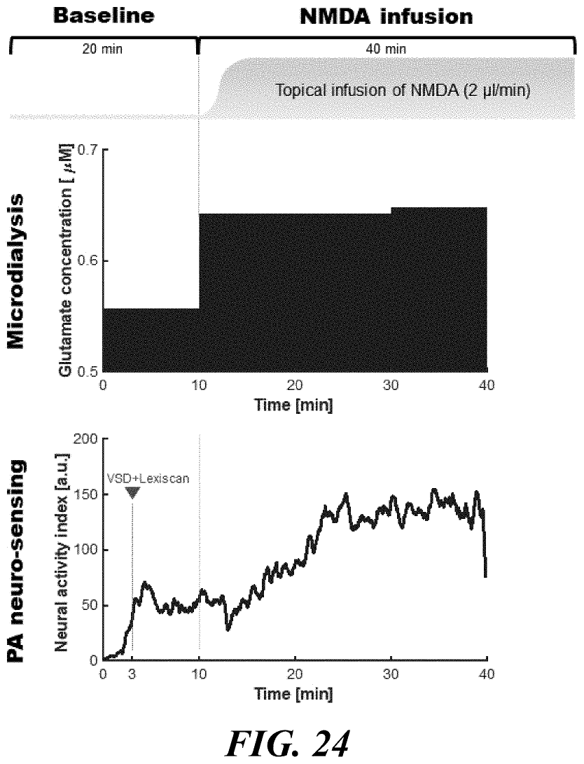

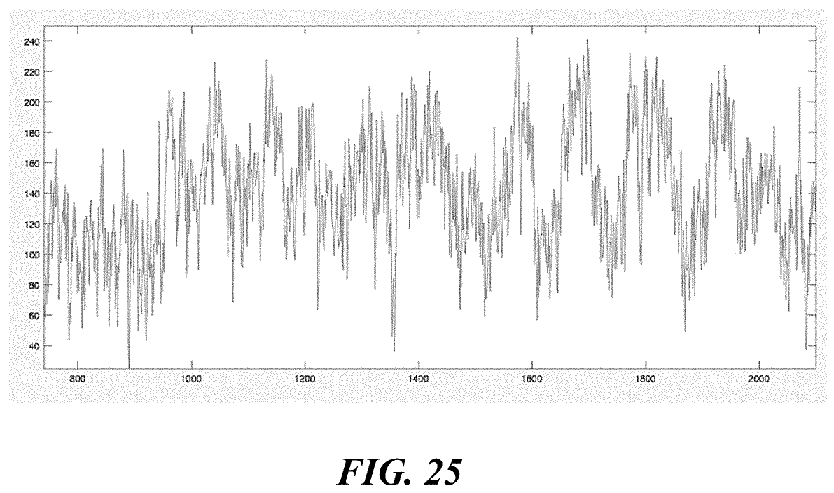

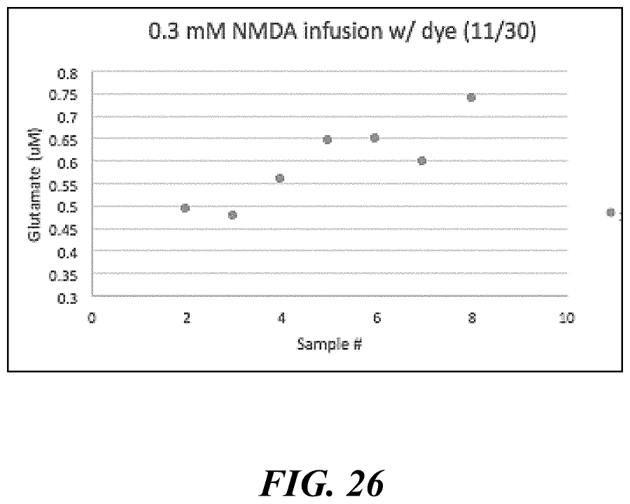

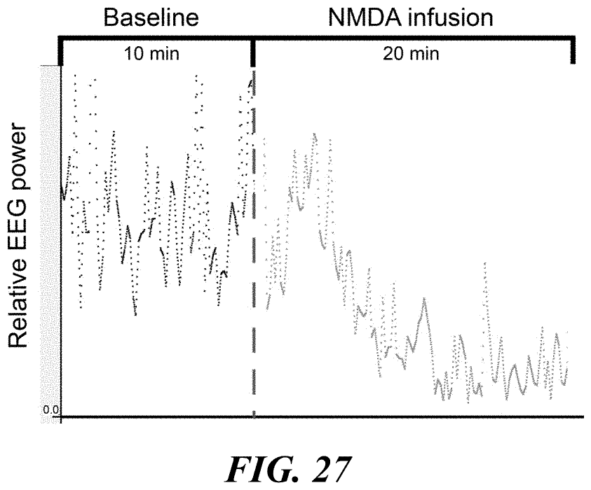

3.1.3 In vivo experimental protocol. The in vivo protocol was designed to have two different phases: baseline and NMDA infusion (FIG. 22B). PA data acquisition was continuously performed for 40 min to be correlated with the glutamate concentration change measured by microdialysis with 20-min intervals. The baseline phase was designed to confirm its stable neural activity before the NMDA infusion. At 3-min time point after starting PA recording, VSD and Lexiscan were administrated through the dosing protocol as follows: through the catheter port located in the tail vein, 150 μl of Lexiscan 0.4 mg/5 ml concentration was injected, and 300 μl of VSD was subsequently administrated at 0.1 mg/ml concentration, followed by 150 μl of saline solution flush. The following 7 min was reserved as the baseline to differentiate the fluctuation of neural activity evoked by NMDA infusion. At 10 min time point, the first microdialysis sample was acquired for the baseline glutamate concentration, and the NMDA infusion was initiated, which evoke the de-synchronized firing of multiple neurons. The subsequent extracellular fluid samples (#2 and #3) were also acquired for the following 40 min, and PA recording was extended up to 30 min from the initiation of NMDA infusion 3.1.4 Results. FIG. 23, FIG. 24, FIG. 25, FIG. 26, and FIG. 27 show the experimental results obtained from the in vivo experiments; two different phases before and after the NMDA infusion have clearly differentiated each other in extracellular glutamate concentration change; In the baseline phase, the glutamate concentration was at 0.557 μM, but it was increased to 0.643 and 0.648 μM at the NMDA infusion phase. The trend was also repeated with the neural activity index estimated by PA recording. After the VSD and Lexiscan administration, the index was stably maintained during baseline phase, but it gradually increases up to 3.27 times with 30 min of NMDA infusion. The increase of index is saturated around 10-15 min of transition from the start of NMDA infusion.

### Example 4

### Visual Stimulation—Visual Cortex Recording

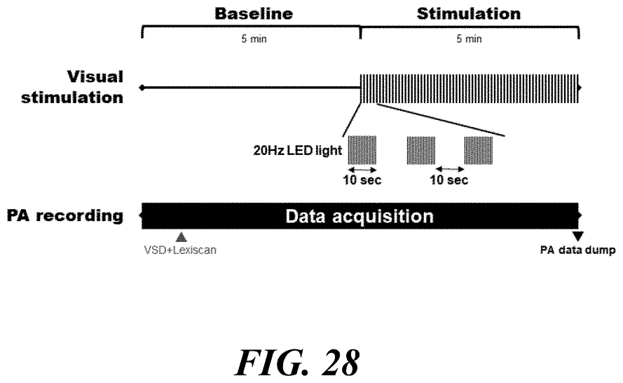

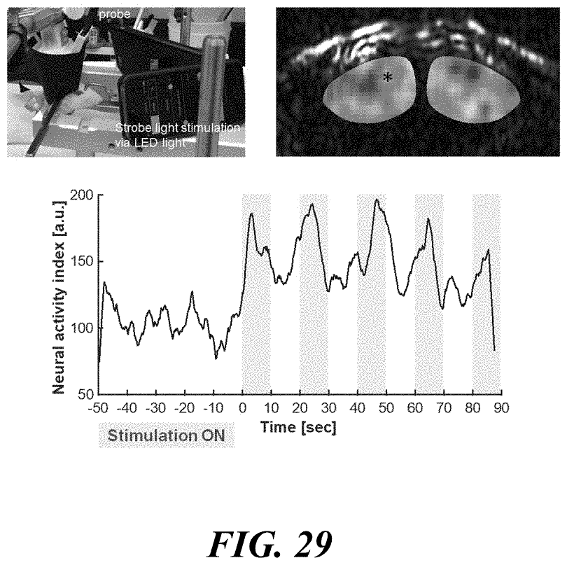

4.1 In vivo experimental protocol. The in vivo protocol was designed to have two different phases: baseline and visual stimulation phase (FIG. 28). PA data acquisition was continuously performed for 10 min. The baseline phase was designed to confirm its stable neural activity before the visual stimulation. At 2-min time point after starting PA recording, VSD and Lexiscan were administrated through the dosing protocol as follows: through the catheter port located in the jugular vein, 150 μl of Lexiscan 0.4 mg/5 ml concentration was injected, and 300 μl of VSD was subsequently administrated at 0.1 mg/ml concentration, followed by 150 μl of saline solution flush. Visual stimulation started at 5 min time point, and it consists of 10-sec of strobe light excitation at 20 Hz in 10-sec interval. The iphone 5s and 6s were used for biocular stimulation by using Flashlight by Rik v.3.0 application. Note that the visual sedation was conducted before starting the experiment for 15 min to stabilize the electrophysiological change at visual cortex of rat brain. Also, interferential stimulation by the 532 nm light coming from the Nd:YAG laser system for imaging was blocked by the laser blocking fabric (FIG. 29A).

4.2 Results. FIG. 29 presents the results from the in vivo visual stimulation experiment. FIG. 29B demonstrates the representative neural activity map projected from 5 min to 10 min, and the asterisk mark indicates the region of interest for neural activity index quantification. FIG. 29C shows the trace of neural activity index, and it shows the elevated activity with corresponding interval of visual stimulation.

### Example 5

### LED-Based Transcranial Photoacoustic Imaging Technology

5.1 In vivo experimental preparation. The translation of the developed technology into clinics can be further facilitated with further reduced cost and guaranteed safety. To obtain these objectives, LED-based transcranial imaging was evaluated, which is expected to be safer and cost-effective, while providing faster imaging speed up to 4 kHz. For the preliminary in vivo validation, a near-infrared pulsed LED illumination system (Prexion Inc., Japan) was used for PA signal generation. The arrays of HDHP LED light source can be comprised of various wavelengths throughout the spectral range of 365 nm to 1450 nm. Especially, near-infrared wavelengths such as 690 nm, 760 nm, 780 nm, and 850 nm can be used for diagnostic PA imaging with deep penetration depth. Also, the system supports combination mode between 690 nm and 850 nm. The effective illumination area of each LED head is 50×7 mm², which is compatible with common clinical ultrasound array transducers. The pulse energy is up to 200 μJ at variable pulse width from 30-135 nm. The pulse repetition rate is up to 4 kHz, which is desirable for high-speed PA imaging. To collect the generated PA signals, ultrasound research package with data acquisition system (SonixTouch and SonixDAQ, Ultrasonix Corp., Canada) was used with a 10-MHz linear ultrasound probe (L14-5/38, Ultrasonix Corp.).

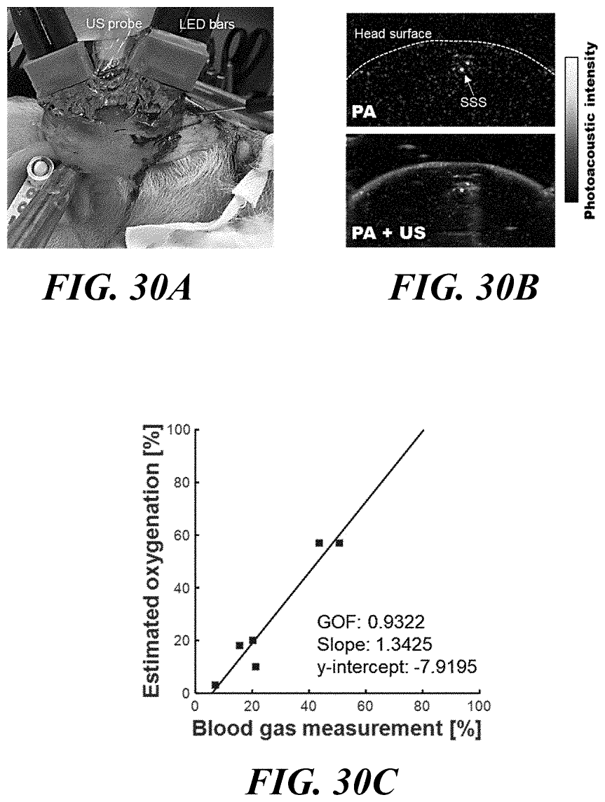

5.2 Results. FIG. 30 shows the preliminary in vivo experimental results of transcranial photoacoustic sensing using LED light source at 850 nm. The substantial amount of photoacoustic imaging could be measured from superior sagittal sinus, which implies its efficacy in clinical diagnosis of hypoxic-ischemic diseases. Customized probe configurations which are optimized for clinical circumstances for fetus and/or newborn babies can be developed. Since LED bars at two wavelengths (i.e., 690 nm and 850 nm) are present, the oxygen saturation at the superior sagittal sinus (SSS) can be estimated as in FIG. 30C; the acceptable goodness of fit (GOF) and y-intercept were obtained (0.93 and −7.91, respectively), but there was more error in slope compared to those in the results with Nd:YAG laser system (34% vs. 5%). This error is thought to be generated due to the estimation at non-optimized wavelengths in the in vivo circumstance, which is obliged with current LED laser system. This process can be further optimized, potentially leading to obtaining clinically-meaningful information from brain tissue regions.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Acker C D, Hoyos E, and Loew L M, “EPSPs measured in proximal dendritic spines of cortical pyramidal neurons,” eNeuro 3(2), 1-13 (2016).
Acker C D, Yan P, and Loew L M, “Single-voxel recording of voltage transients in dendritic spines,” Biophys. J. 101(2), L11-L13 (2011).
Adler D A, et al. (2014) Circadian cycle-dependent EEG biomarkers of pathogenicity in adult mice following prenatal exposure to in utero inflammation. Neuroscience 275:305-313.
American National Standard for the Safe Use of Lasers. (1975) American National Standard for the Safe Use of Lasers. Annals of Internal Medicine 82(1):132.
Arthurs O J and Boniface S, “How well do we understand the neural origins of the fMRI BOLD signal?” Trends Neurosci. 25(1), 27-31 (2002).
Arthurs O J et al., “Intracortically distributed neurovascular coupling relationships within and between human somatosensory cortices,” Cereb. Cortex 17(3), 661-668 (2007).
Berman M G, Jonides J, Nee D E (2006) Studying mind and brain with fMRI. Social cognitive and affective neuroscience 1(2):158-161.
Canepari M, Bemus O, and Zecevic D, Membrane Potential Imaging in the Nervous System and Heart, p. 516, Springer, New York (2015).
Carman A J, Mills J H, Krenz A, Kim D-G, Bynoe M S (2011) Adenosine receptor signaling modulates permeability of the blood-brain barrier. Journal of Neuroscience 31(37):13272-13280.
Chen H and Diebold G, “Chemical generation of acoustic waves: a giant photoacoustic effect,” Science 270(5238), 963-966 (1995).
Chu P-C, et al. (2015) Neuromodulation accompanying focused ultrasound-induced blood-brain barrier opening. Sci Rep 5:15477.
Deán-Ben X L, et al. (2016) Functional optoacoustic neuro-tomography for scalable whole-brain monitoring of calcium indicators. Light Science Applications 5(12):e16201-7.
Devor A, et al. (2012) Frontiers in optical imaging of cerebral blood flow and metabolism. J Cereb Blood Flow Metab 32(7):1259-1276.
Friston K J (2009) Modalities, Modes, and Models in Functional Neuroimaging. Science 326(5951):399-403.
Frost J J (2003) Molecular imaging of the brain: A historical perspective. Neuroimaging Clinics of North America 13(4):653-658.
Goldman H, Berman R F, Hazlett H, Murphy S (1992) Cerebrovascular responses to pentylenetetrazol: time and dose dependent effects. Epilepsy Res 12:227-242.
Grillner S, et al. (2016) Worldwide initiatives to advance brain research. Nat Neurosci 19(9):1118-1122.
Heo C, et al. (2016) A soft, transparent, freely accessible cranial window for chronic imaging and electrophysiology. Sci Rep 6(1):27818.
Hillman E M C (2007) Optical brain imaging in vivo: techniques and applications from animal to man. J Biomed Opt 12(5):051402-051402-28.
Hoshi Y, Tamura M (1993) Dynamic changes in cerebral oxygenation in chemically induced seizures in rats: study by near-infrared spectrophotometry. Brain Res 603(2):215-221.
Hu S, “Listening to the brain with photoacoustics,” IEEE J. Sel. Top. Quantum Electron. 22(3), 117-126 (2016).
Johnston M V, et al. (2014) Twenty-four hour quantitative-EEG and in-vivo glutamate biosensor detects activity and circadian rhythm dependent biomarkers of pathogenesis in Mecp2 null mice. Front Syst Neurosci 8(June):118.
Lewis P M, Thomson R H, Rosenfeld J V, Fitzgerald P B (2016) Brain Neuromodulation Techniques: A Review. Neuroscientist:1073858416646707.
Loew L M et al., “Fluorometric analysis of transferable membrane pores,” Biochemistry 24(9), 2101-2104 (1985).
Loew L M, “Design and use of organic voltage sensitive dyes,” Adv. Exp. Med. Biol. 859, 27-53 (2015).
Logothetis N K (2008) What we can do and what we cannot do with fMRI-ProQuest. Nature.
Löscher W (2017) Animal Models of Seizures and Epilepsy: Past, Present, and Future Role for the Discovery of Antiseizure Drugs. Neurochemical Research 0(0):0-0.
Luke G P, Yeager D, and Emelianov S Y, “Biomedical applications of photoacoustic imaging with exogenous contrast agents,” Ann. Biomed. Eng. 40(2), 422-437 (2012).
Mantini D, Perrucci M G, Del Gratta C, Romani G L, Corbetta M (2007) Electrophysiological signatures of resting state networks in the human brain. Proc Natl Acad Sci USA 104(32):13170-13175.
Martišienė I, et al. (2016) Voltage-Sensitive Fluorescence of Indocyanine Green in the Heart. BPJ 110(3):723-732.
McArthur E A et al., “A study of the binding of cyanine dyes to colloidal quantum dots using spectral signatures of dye aggregation,” J. Phys. Chem. C 116(10), 6136-6142 (2012).
Mujumdar R B et al., “Cyanine dye labeling reagents: sulfoindocyanine succinimidyl esters,” Bioconjugate Chem. 4(2), 105-111 (1993).
Nasiriavanaki M et al., “High-resolution photoacoustic tomography of resting-state functional connectivity in the mouse brain,” Proc. Natl. Acad. Sci. U.S.A 111(1), 21-26 (2014).
Nehlig A, et al. (1996) Absence seizures induce a decrease in cerebral blood flow: Human and animal data. Journal of Cerebral Blood Flow and Metabolism 16(1):147-155.
Nowak K, et al. (2011) Optimizing a Rodent Model of Parkinson's Disease for Exploring the Effects and Mechanisms of Deep Brain Stimulation. Parkinson's Disease 2011(2754):1-19.
Onoe S, Temma T, Shimizu Y, Ono M, Saji H (2014) Investigation of cyanine dyes for in vivo optical imaging of altered mitochondrial membrane potential in tumors. Cancer Med 3(4):775-786.
Paxinos G, Watson C (2014) The Rat Brain in Stereotaxic Coordinates (Elsevier Academic Press). Seventh edition Available at: https://books.google.com/books?id=FuqGAwAAQBAJ&printsec=frontcover#v=one
Qin H et al., “Fluorescence quenching nanoprobes dedicated to in vivo photoacoustic imaging and high-efficient tumor therapy in deep-seated tissue,” Small 11(22), 2675-2686 (2015).
Raichle M E (1998) Behind the scenes of functional brain imaging: a historical and physiological perspective. Proc Natl Acad Sci USA 95(3):765-772.
Raichle M E, Mintun M A (2006) Brain Work and Brain Imaging. http://dxdoiorg/101146/annurevneuro29051605112819 29(1):449-476.
Rao B, Zhang R, Li L, Shao J-Y, Wang L V (2017) Photoacoustic imaging of voltage responses beyond the optical diffusion limit. Sci Rep 7(1):S1-10.
Roland P E, Hilgetag C C, Deco G (2014) Cortico-cortical communication dynamics. Front Syst Neurosci 8:19.
Sarimollaoglu M et al., “Nonlinear photoacoustic signal amplification from single targets in absorption background,” Photoacoustics 2(1), 1-11 (2014).
Sims P J et al., “Studies on the mechanism by which cyanine dyes measure membrane potential in red blood cells and phosphotidylcholine,” Biochemistry 13(16), 3315-3330 (1974).
Sinha S H et al., “Synthesis and evaluation of carbocyanine dyes as PRMT inhibitors and imaging agents,” Eur. J. Med. Chem. 54, 647-659 (2012).
Strangman G E, Li Z, Zhang Q (2013) Depth sensitivity and source-detector separations for near infrared spectroscopy based on the Colin27 brain template. PLoS ONE 8(8):e66319.
Torricelli A, et al. (2014) Time domain functional NIRS imaging for human brain mapping. NeuroImage 85 Pt 1:28-50.
Sun, T, Zhang, Y, Power, C, Alexander, P M, Sutton, J T, Aryal, M, Vykhodtseva, N, Miller, E L, and McDannold, N J, PNAS Plus: Closed-loop control of targeted ultrasound drug delivery across the blood-brain/tumor barriers in a rat glioma model, PNAS 2017 114 (48) E10281-E10290; published ahead of print Nov. 13, 2017.
Treger J S, Priest M F, Iezzi R, Bezanilla F (2014) Real-time imaging of electrical signals with an infrared FDA-approved dye. Biophysj 107(6):L09-L012.
Tufail Y, Yoshihiro A, Pati S, Li M M, Tyler W J (2011) Ultrasonic neuromodulation by brain stimulation with transcranial ultrasound. Nat Protoc 6(9):1453-1470.
Vanitha N V (2011) Positron emission tomography in neuroscience research. Annals of Neurosciences 18(2):36.
Walther A et al., “Two-photon voltage imaging using a genetically encoded voltage indicator,” Sci. Rep. 3, 2231 (2013).
Wang L V and Hu S, “Photoacoustic tomography: in vivo imaging from organelles to organs,” Science 335(6075), 1458-1462 (2012).
Wang L V, “Multiscale photoacoustic microscopy and computed tomography,” Nat. Photonics 3, 503-509 (2009).
Wang X, et al. (2003) Noninvasive laser-induced photoacoustic tomography for structural and functional in vivo imaging of the brain. Nat Biotechnol 21(7):803-806.
Weber J, Beard P C, and Bohndiek S E, “Contrast agents for molecular photoacoustic imaging,” Nat. Methods 13, 639-650 (2016).
Wu D et al., “Contrast agents for photoacoustic and thermoacoustic imaging: a review,” Int. J. Mol. Sci. 15(12), 23616 (2014).
Yan P et al., “A palette of fluorinated voltage sensitive hemicyanine dyes,” Proc. Natl. Acad. Sci. U.S.A 109(50), 20443-20448 (2012).
Yao J and Wang L V, “Photoacoustic brain imaging: from microscopic to macroscopic scales,” Neurophotonics 1(1), 011003 (2014).
Zhang H K, et al. (2017) Listening to membrane potential: photoacoustic voltage-sensitive dye recording. J Biomed Opt 22(4):045006.
Zhang T, et al. (2014) Pre-seizure state identified by diffuse optical tomography. Sci Rep 4(1):113-10.

Although the foregoing subject matter has been described in some detail by way of illustration and example for purposes of clarity of understanding, it will be understood by those skilled in the art that certain changes and modifications can be practiced within the scope of the appended claims.

1-12. (canceled)

13. A method for measuring an electrophysiological activity in a subject in vivo, the method comprising administering one or more photoacoustic voltage dependent dyes of formula (I) to a target area of the subject: 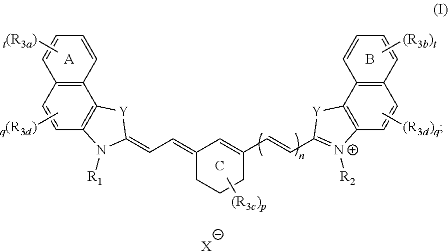
wherein:
n is an integer selected from 0, 1, and 2; p and t are each independently integers selected from 1, 2, 3, and 4; q is an integer selected from 1 or 2; A and B can be present or absent; C is a ring that can be present or absent and when present forms part of the polymethine chain, wherein ring C or the polymethine chain of which it forms a part of can be substituted with R_{3c}; R₁ and R₂ can be the same or different and are each independently selected from alkyl, substituted alkyl, and —(CH₂CH₂O)ₘ—R₄, wherein m is an integer from 1 to 20 and R₄ is selected from the group consisting of H, alkyl, and —OR₅, wherein R₅ is alkyl; each R₃ₐ, R_{3b}, R_{3c}, and R_{3d} is selected from the group consisting of hydrogen, alkyl, halogen, hydroxyl, cyano, and alkoxyl; Y is selected from O, S, and C(R₆)₂, wherein each R₆ is independently H or alkyl; X is a counterion; and pharmaceutically acceptable salts thereof;
irradiating the target area of the subject with near-infrared radiation; and
measuring a photoacoustic signal from the target area of the subject, wherein the photoacoustic signal is indicative of the electrophysiological activity in the subject.

14. The method of claim 13, wherein the administering of the one or more photoacoustic voltage dependent dyes of formula (I) includes co-administering the dye with an agent capable of pharmacological modulation of adenosine receptor signaling, incorporating the dye in a brain-penetrating nanoparticle, incorporating the dye in a microbubble, focused ultrasound, and combinations thereof.

15. The method of claim 13, wherein the detecting of the photoacoustic signal comprises ultrasound detection.

16. The method of claim 13, wherein the target area of the subject comprises an organ of the subject.

17. The method of claim 16, wherein the organ is selected from the group consisting of the brain, heart, kidney, liver, and muscle.

18. The method of claim 13, wherein the method measures a potential change in an organ, tissue, or cell of the subject.

19. An integrated photoacoustic imaging system for measuring an electrophysiological activity in a subject in vivo, the system comprising:
(a) a near-infrared light source;
(b) an ultrasound probe; and
(c) a data acquisition system.

20. The integrated photoacoustic imaging system of claim 19, wherein the near-infrared light source comprises a tunable laser.
